(19) United States
(12) Patent Application Publication
(10) Pub. No.: US 2004/0198819 A1
(43) Pub. Date: Oct. 7, 2004

(54) Biological response modifier composition and uses thereof

(76) Inventor: Aiping H. Young, North York Ontario (CA)

Correspondence Address: Knoble & Yoshida, Eight Penn Center, Suite 1350, 1628 John F Kennedy Boulevard, Philadelphia, PA, 19103, US

(21) Appl. No.: 10/481,867

(22) PCT Filed: Jun. 20, 2002
(86) PCT No.: PCT/CA02/00932

(30) Foreign Application Priority Data
Jun. 20, 2001 (CA) 2,351,169
Apr. 8, 2002 (CA) 2,380,967

(51) Int. Cl. A61K 31/185; A61K 31/19; A61K 31/198; A61K 31/17

(52) U.S. Cl. 514/557; 514/561; 514/553; 514/588; 514/642

(57) ABSTRACT

The present invention provides synthetic biological response modifier compositions. The synthetic biologic response modifier (Syn-BRM) composition comprises: 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea. The compositions may additionally comprise 40±14 mg/L of formic acid. The synthetic biological response modifier compositions modify biological response in vivo demonstrating anti-cancer activity and enhancing cell-mediated immune response to tumours. The invention further provides anticancer combinations comprising a synthetic biological response modifier composition and one or more anticancer agents, wherein said combination improves the treatment of cancers over the composition or the anticancer agents alone. Another aspect of the present invention provides the use of synthetic biological response modifier compositions or an anticancer combination thereof in the manufacture of a medicament or a pharmaceutical kit and in the treatment of cancer.

### FIELD OF THE INVENTION

The present invention relates to a synthetic biological response modifier composition, pharmaceutical compositions comprising the same and the uses thereof, alone and in combination with other drugs in the treatment of various disorders.

### BACKGROUND OF THE INVENTION

Numerous therapies exist that are directed towards the treatment of cancer, viral disorders, and many other diseases including chemotherapeutic drugs, radiation, gene therapy and antisense oligonucleotides. One drawback to current therapies is the toxic side effects associated with the pharmaceuticals utilized to treat different human diseases. Moreover, oftentimes large dosages must be administered over an extended period of time in order to attain therapeutic benefit. Thus, a need remains for more effective therapeutic treatments for human disease.

Therapies are continuously being developed for the prophylaxis and treatment of cancer and infectious diseases, such as Acquired Immunodeficiency Syndrome (AIDS). Some of these therapies attempt to use the immune system therapeutically. One approach is based on the antigen specific elements of the immune system, namely antibodies and T cells. For example, research has been aimed at developing vaccines against foreign agents, or against certain endogenous chemical messengers, such as interleukins, to suppress antibody reactions. A second approach is based on the isolation, cloning, expression and production of peptides and proteins from the non-antigen specific parts of the immune system. For example, proteins, such as cytokines, which comprise the interleukins produced by white blood cells, and interferons which stimulate lymphocytes and scavengers cells that digest foreign antigens, offer possibilities for therapies.

The treatment of many human diseases could be greatly enhanced if the early immune response to a foreign antigenic molecule, substance, cell or organism could be augmented so that the immune response of the affected individual is enhanced. Strategies which have been suggested to augment the immune response include vaccines specific for disease-associated antigens; the use of monoclonal antibodies against antigens on the surface of cells or viruses and superantigens.

Relatively recently, the role of the physiologically active polypeptide, known as tumor necrosis factor (“TNF”) has been studied, particularly with respect to its ability to induce necrosis of tumors, with no effect upon the normal tissues of the living body. The amino acid sequence of TNF, as well as the base sequence of the DNA coding for TNF has been disclosed in U.S. Pat. No. 4,879,226.

Because TNF has been shown to have a role in controlling the immune system of mammals in response to a variety of different diseases, any agent that can stimulate the production or bioavailability of TNF in vivo has potential utility as a treatment for various human diseases, including cancer, HIV, heart disease and others. Additionally, any agent that can stimulate human monocytes and macrophages to produce TNF in vitro, is useful as a means for providing a source of TNF for therapeutic administration, as well as for analytical and diagnostic purposes.

The mechanism of action of TNFα appears to be derived from accumulating evidence which indicates that TNFα is a regulatory cytokine with pleiotrophic biological activities. These activities include: inhibition of lipoprotein lipase synthesis, activation of polymorphonuclear leukocytes, inhibition of cell growth or stimulation of cell growth, cytotoxic action on certain transformed cell lines, antiviral activity, stimulation of bone resorption, stimulation of collagenase and prostaglandin E2 production and immunoregulatory actions including activation of T-cells, B-cells, monocytes, thymocytes and stimulation of the cell-surface expression of major histocompatibility complex class I and class II molecules.

Unfortunately, treatments with high dosages of TNF alone have been associated with such side effects as hypotension, leukocytosis, fever, chills, neurotoxicity, nausea and vomiting. TNF therapy has therefore played an important role in the field of cancer therapy, however excessive or unregulated TNF production has been implicated in exacerbating a number of disease states. These include rheumatoid arthritis, rheumatoid spondylitis, osteoarthritis, gouty arthritis and other arthritic conditions, sepsis, septic shock, gram negative sepsis, toxic shock syndrome, adult resipratory stress syndrome, cerebral malaria, chronic pulmonary inflammatory disease, silicosis, pulmonary sarcoidosis, bone resorption disease, reperfusion injury, graft v. host reaction, allograft rejections, fever and myalgias due to infection, such as influenza, cachexia secondary to infection or malignancy, cachexia secondary to AIDS, keloid formation, scar tissue formation, Crohn's disease, ulcerative colitis and pyresis plus a number of autoimmune diseases such as multiple sclerosis, autoimmune diabetes and systemic lupus erythematosis.

Cytokines, specifically TNF, have been implicated in the activation of T-cell mediated HIV protein expression and/or virus replication by playing a role in maintaining T-lymphocyte activation. Therefore, extensive research has been directed towards interfering with cytokine production, notably TNF, in a HIV-infected individual. The therapeutic aim being to limit the maintenance of T-cell activation, thereby reducing the progression of HIV infectivity to previously uninfected cells, thereby resulting in a slowing or elimination of the progression of immune dysfunction caused by HIV infection. Hence there is mounting evidence supporting the use of inhibitors of cytokines, particularly TNF, (U.S. Pat. Nos. 5,563,143 and 5,506,340) in the treatment of AIDS.

Numerous clinical trials have also been carried out in patients with Kaposi's sarcoma with immune modulators such as Interferonα (J. AIDS., 1:111-118; 1988). This drug has been licensed in Canada for the treatment of Kaposi's sarcoma. Interferon has been shown to have antitumor and antiretroviral effects. Response rates to treatment with IFN are initially high (Krown, et al., Recomb. Leucocyte A IFN in Kaposi's sarcoma, N.Y. Acad. Sci., 437:431-438, 1984). However prolonged responses are not frequent, possibly because of the emergence of anti-IFN antibodies (Autavelli, et al., J.I.D. 163:882-885, 1991). Patients invariably require chemotherapy or radiotherapy to control tumor growth. Both IFN and chemotherapy have substantial toxic side effects on bone marrow resulting in the termination of therapy (Fischl, M. A., Am. J. Med., Apr. 10, 1991).

Both TNF and IFN individually possess antiviral activity, making them potential candidates in the treatment of viral infections and tumors. However, serious side effects have been observed in the treatment with therapeutically valuable doses of TNF and IFN which have limited their clinical usefulness.

Infectious diseases, such as those caused by viruses can only succeed by avoiding or defeating the body's immune system. The immune system mounts or elicits either or both non-specific immune responses and specific immune response factors to fight such pathogens.

Non-specific immune responses are focused on cytokine production, principally by macrophages, and serve as a prelude to specific antibody responses. The inflammatory cytokines include TNF-α and mediate an acute response directed to the injury or infection sites, which is manifested by an increased blood supply. The pathogenic bacteria or viruses are engulfed by neutrophils and macrophages in an attempt to contain the infection to a small tissue space. Macrophages, therefore, play a key role in the defense against infectious diseases as follows: (1) processing and presentation of antigens to lymphocytes so that antibody-mediated and cell-mediated immune responses can occur; (2) secretion of cytokines central to immune response; and (3) destruction of antibody-coated bacteria, tumor cells or host cells.

Macrophages can ingest and kill a wide variety of pathogens, such as bacteria, fungi, and protozoa (parasites). This ability is augmented when the macrophages are activated. Secreted products of activated macrophages are more diverse than those from any other immune cell. These regulate both pro- and anti-inflammatory effects and regulate other cell types. These products include TNF-α, IL-1β, IL-6, hydrolytic enzymes, and products of oxidative metabolism Bacteria that are eliminated primarily through this cell-mediated immune process include tuberculosis and other related mycobacterial infections, such as a typical mycobacterial infections seen in up to 50% of AIDS patients, and anthrax, a potential bacteriological warfare agent. Fungal infections are common problems in immunosuppressed patients, such as those afflicted with AIDS or organ transplant patients.

Therefore a need remains for a synthetic BRM (Syn-BRM) that alone or in combination with other pharmaceuticals modulates the mammalian immune system in a manner that effectively treats human disease.

### SUMMARY OF THE INVENTION

The invention provides a synthetic biological response modifier (Syn-BRM) composition.

In accordance with an aspect of the present invention there is provided a synthetic biological response modifier composition comprising 57±20 mg/L 3-hydroxybutyric acid, 125±44 mg/L lactic acid, 155±54 mg/L acetic acid, 1.4+0.5 mg/L creatine, 22±8 mg/L creatinine, 2.5±0.9 mg/L carnitine, 6.8±2.4 mg/L taurine, 20±7 mg/L choline, 815±285 mg/L urea, wherein said composition: (a) stimulates or activates monocytes and macrophages; and/or (b) modulates tumor necrosis factor production and/or release.

In accordance with another aspect of the present invention there is provided a synthetic biological response modifier composition comprising 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea, 40±14 mg/L formic acid, wherein said composition stimulates or activates monocytes and macrophages; and/or modulates tumor necrosis factor production and/or release.

In accordance with another aspect of invention there is provided a synthetic biological response modifier composition comprising 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea, 40±14 mg/L and the relative proportions of the components in the composition remains approximately the same.

In accordance with a further aspect of the invention there is provided a combination comprising: a composition of the invention and one or more anticancer agent(s), wherein said combination has therapeutic synergy or improves the therapeutic index in the treatment of cancer over the composition or the anticancer agent(s) alone.

The invention also encompasses pharmaceutical compositions comprising the synthetic biologic response modifier compositions of the invention.

The present invention encompasses a method of treating a mammal (or patient) comprising the step of administering to said mammal an effective amount of a synthetic biological response modifier composition, either alone or in combination with one or more anti-cancer drugs and/or anti-viral drugs and/or antisense sequences, as well as to pharmaceutical compositions and kits comprising a combination of the synthetic biological response modifier composition and anticancer drugs, anti-virals and/or antisense sequences.

These and other aspects of the present invention will become evident upon reference to the following detailed description and attached drawings.

### BRIEF DESCRIPTION OF THE DRAWINGS

Further details of the invention are described below with the help of the examples illustrated in the accompanying drawings in which:

FIG. 1 presents results of an in vivo demonstration of the effect of two embodiments of a synthetic BRM composition on the tumors of human pancreatic carcinoma (BxPC-3) in CD1 nude mice. FIG. 1A demonstrates the effect on tumor size and FIG. 1B demonstrates the effect on tumor weight.

FIG. 2 presents results of an in vivo demonstration of the effect of two embodiments of a synthetic BRM composition on the tumors of human pancreatic carcinoma (BxPC-3) in CD-1 nude mice, compared to a natural BRM. FIG. 2A demonstrates the effect on tumor size and FIG. 2B demonstrates the effect on tumor weight.

FIG. 3 presents results of an in vivo demonstration of the effect of two embodiments of a synthetic BRM composition on the tumors of human melanoma (C8161) in CD-1 nude mice, compared to a natural BRM. FIG. 3A demonstrates the effect on tumor size and FIG. 3B demonstrates the effect on tumor weight

FIG. 4 presents results of an in vivo demonstration of the effect of two embodiments of a synthetic BRM composition on the tumors of human breast adenocarcinoma (MDA-MB-231) in CD-1 nude mice, compared to a natural BRM. FIG. 4A demonstrates the effect on tumor size and FIG. 4B demonstrates the effect on tumor weight

FIG. 5 presents results of an in vivo demonstration of the effect of two embodiments of a synthetic BRM composition on the tumors of human pancreatic carcinoma (BxPC-3) in CD-1 nude mice, compared to a natural BRM. FIG. 5A demonstrates the effect on tumor size and FIG. 5B demonstrates the effect on tumor weight

FIG. 6 presents results of an in vivo demonstration of the effect of two embodiments of a synthetic BRM composition on the tumors of human breast adenocarcinoma (MDA-MB-231) in CD-1 nude mice, compared to a natural BRM. FIG. 6A demonstrates the effect on tumor size and FIG. 6B demonstrates the effect on tumor weight.

FIG. 7A presents results of an in vivo demonstration of the effect of a synthetic BRM-composition on the weight of human pancreatic carcinoma tumors (BxPC-3) in CD-1 nude mice, compared to a natural BRM.

FIG. 7B demonstrates the effect of a synthetic BRM composition on TNF-α release.

FIG. 8 shows the effect of syn-BRM on NK cell infiltration into tumors in mice harboring Human Melanoma (C8161) xenografts.

FIG. 9 illustrates a 2D COSY spectrum of Nat-BRM with various J-coupling connections of known compounds labeled in the spectrum.

FIG. 10 illustrates a 2D NOESY spectrum of Nat-BRM with various NOE connections of known compounds labeled in the spectrum.

FIG. 11 illustrates a 1D NMR spectrum of Nat-BRM with added formic acid (18A+Formic acid) and Nat-BRM (18A) alone.

FIG. 14 illustrates a 1D proton NMR spectrum of Nat-BRM with chemical shift assignments for identified compounds at, a) 8.1-8.8 ppm, b) 2.74.4 ppm, and c) 0.8-2.6 ppm.

FIG. 16 compares the percentage of inhibition of tumor weight by Syn-BRM (Syn-BRM#4) and various batches of Nat-BRM in human carcinoma models. Inhibition of tumor weight by Nat-BRM was calculated using the formula [(C−T)/C]×100, where C represents mean tumor weight of saline-treated mice and T represents that of Nat-BRM-treated mice. Each bar represents the calculated percentage of tumor weight inhibition (mean±SE) for the treatment groups. The total number of experiments performed per treatment group is indicated on the right.

FIG. 17 illustrates the effects of Nat-BRM treatment on numbers of tumor infiltrating NK cells. (A) Mice harboring C8161 human melanoma xenografts were treated with saline or Nat-BRM and isolated tumors were subjected to FACS analyses using macrophage- and NK cell-specific antibodies. Increased NK infiltration (6.45% to 10.55%) to tumors isolated from mice treated with Nat-BRM was observed. (B) Mice harboring Capan-1 human pancreatic carcinoma xenografts were treated with saline or Nat-BRM and isolated tumors were subjected to flow cytometric analyses using macrophage- and NK cell-specific antibodies. Increased NK infiltration (4.49% to 9.70%) to tumors isolated from mice treated with Nat-BRM was observed.

FIG. 18 illustrates the effects of macrophage depletion on in vivo anti-tumor efficacy of Nat-BRM. (A) the effects on tumor size and (B) the effects on tumor weight.

FIG. 19 illustrates the effects of Nat-BRM on NK cell infiltration to tumors in macrophage depleted mice. CD-1 nude mice harboring C8161 human melanoma xenografts were depleted of macrophages using C12MDP and treated with saline control or Nat-BRM. NK infiltration is significantly compromised in tumors isolated from macrophage-depleted mice, suggesting an important role of macrophages in Nat-BRM-mediated NK infiltration.

FIG. 20 illustrates the growth of Human Pancreatic Adenocarcinoma (BxPC-3) in CD-1 Nude Mice.

FIG. 21 illustrates the weight of Human Pancreatic Adenocarcinoma (BxPC-3) in CD-1 Nude Mice.

FIG. 22 illustrates the growth of Human Pancreatic Carcinoma (SU.86.86.) in CD-1 Nude Mice.

FIG. 23 illustrates the weight of Human Pancreatic Carcinoma (SU.86.86.) in CD-1 Nude Mice.

FIG. 24 illustrates the growth of Human Melanoma (A2058) in CD-1 Nude Mice.

FIG. 25 illustrates the weight of Human Melanoma (A2058) in CD-1 Nude Mice.

FIG. 26 illustrates the growth of Human Melanoma (C8161) in CD-1 Nude Mice.

FIG. 27 illustrates the weight of Human Melanoma (C8161) in CD-1 Nude Mice.

FIG. 28 illustrates the growth of Human Breast Adenocarcinoma (MDA-MB-231) in CD-1 Nude Mice.

FIG. 29 illustrates the weight of Human Breast Adenocarcinoma (MDA-MB-231) in CD-1 Nude Mice.

FIG. 30 illustrates the growth of Human Breast Adenocarcinoma (MDA-MB-231) in CD-1 Nude Mice.

FIG. 31 illustrates the weight of Human Breast Adenocarcinoma (MDA-MB-231) in CD-1 Nude Mice.

FIG. 32 illustrates the growth of Human Prostate Carcinoma (PC-3) in SCID Mice.

FIG. 33 illustrates the weight of Human Prostate Carcinoma (PC-3) in SCID Mice.

FIG. 34 illustrates the growth of Human Pancreatic Carcinoma (13×PC-3) in CD-1 Nude Mice.

FIG. 35 illustrates the weight of Human Pancreatic Carcinoma (SU.86.86) in CD-1 Nude Mice.

FIG. 36 illustrates the growth of Human Prostate Carcinoma (DU145) in SCID Mice.

FIG. 37 illustrates the weight of Human Prostate Carcinoma (DU145) in SCID Mice.

FIG. 38 illustrates the growth of Human Ovary Adenocarcinoma (SK-OV-3) in CD-1 Nude Mice.

FIG. 39 illustrates the growth of Human Ovary Adenocarcinoma (SK-OV-3) in CD-1 Nude Mice.

FIG. 40 illustrates the growth of Human Lung Adenocarcinoma (H460) in CD-1 Nude Mice.

FIG. 41 illustrates the weight of Human Lung Adenocarcinoma (H460) in CD-1 Nude Mice.

FIG. 42 illustrates the growth of Human Small Cell Lung Carcinoma (1209) in SCID Mice.

FIG. 43 illustrates the weight of Human Small Cell Lung Carcinoma (H209) in SCID Mice.

FIG. 44 illustrates the growth of human melanoma (C8161) in CD-1 Nude Mice. Human melanoma cells were injected into the right flanks of CD-1 nude mice to induce tumor growth. Treatments with saline, defined osmolarity solutions, Nat-BRM and Syn-BRM were started several days following tumor cell inoculation when the tumor had reached a palpable size. Tumor volume was measured by calipers on the indicated days.

Table 1: A synthetic biologic response modifier composition (Syn-BRM #1) of the present invention.

Table 2: A synthetic biologic response modifier composition (Syn-BRM #2) of the present invention.

Table 3: Illustrates further embodiments of the synthetic biologic response modifier composition (Table 3A: Syn-BRM #3 and Table 3B: Syn-BRM#4) of the present invention. Where present, formic acid may be present in the concentration range of 40-107 mg/L.

Table 4: Shows proton chemical shift assignments from NMR spectra of Nat-BRM. b=peak overlapped with other compound peaks. n=not observed in the spectra due to low concentrations.

Table 6: Summary of Inorganic components detected in Nat-BRM.

Table 7: Summary of Nat-BRM in vivo anti-tumour activity assessed in mice harbouring human cancer xenoplants. (N/A) not done; (+) significant tumor growth suppression (P<0.05); (−) no tumor growth suppression; (+/−) variable in different experiments; (+) suppression of tumor growth statistically greater than either treatment alone (P<0.05); (−) suppression of tumor growth not greater than either treatment alone; (+/−) variable in different experiments; (*) tumor suppression greater than either treatment alone but not statistically different

### DETAILED DESCRIPTION OF THE INVENTION

The present invention is a synthetic biologic response modifier (Syn-BRM) comprising components in the following amounts: 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea The compositions may additionally comprise 40±14 mg/L of formic acid. The Syn-BRM been shown to modify biological response in vivo. In one example, the Syn-BRM demonstrates anti-cancer activity and enhances cell-mediated immune response to tumours.

In an embodiment of the invention the compositions may additionally comprise inorganic components including sodium, phoshphate and chloride (as described in the tables and examples) in amounts (+35%) of 14900 mg/L of NaCl, 1390 mg/L of Na₂HPO₄ and 780 mg/L of NaH₂PO₄.

The compositions of the present invention can be made according to known and standard methods in the art. For example, commercially available, optionally pharmaceutical grade compounds or components can be mixed and solubilized with sterile water and/or using appropriate buffers. One skilled in the art would appreciate how to adjust, for example, the osmolarity and pH of the compositions so that the compositions are physiologically acceptable.

For example, the pH of the composition may be adjusted to physiological pH, i.e. 7.4-7.5, using hydrochloric acid (1%) solution and sodium hydroxide (1% solution), and a buffered solution may be obtained using dibasic and monobasic sodium phosphate salts as buffers, using conventional methods. In an embodiment of the invention the pH is 7.0.

In an embodiment of the invention an osmolarity of about 650 mOsm, but may be as high as 850 mOsm.

The composition of the invention can modulate tumor necrosis factor (TNF) production and/or release. In one embodiment a composition of the invention, promotes the release of TNF from human peripheral blood mononuclear cells and from the pre-monocyte cell line U-937. As TNF is known to initiate a cascade of inflammatory and antitumor cytokine effects, one embodiment stimulates human leukocytes to release TNF and other cytokines including those listed in Example 22.

Once prepared, therefore, an embodiment of the composition it can be tested for its ability to stimulate TNF release, for example, using methodologies as taught in Example 8. The compositions activate PBMNs to release TNF in vitro as measured by the Monocyte/Macrophage Activation Assay (TNF-Release).

The efficacy of the compositions of the present invention may be determined experimentally using standard techniques using cancer models well known to workers skilled in the art. Such cancer models allow the activity of combinations to be tested in vitro and in vivo in relation to the cancer of interest. Exemplary methods of testing activity are described in the Examples provided herein, although, it should be understood that these methods are not intended to limit the present invention.

One example of a method for studying the efficacy of the compositions on solid tumors in vivo involves the use of subject animals, generally mice, that are subcutaneously grafted bilaterally with 30 to 60 mg of a tumor fragment on day 0. The animals bearing tumors are mixed before being subjected to the various treatments and controls. In the case of treatment of advanced tumors, tumors are allowed to develop to the desired size, animals having insufficiently developed tumors being eliminated. The selected animals are distributed at random to undergo the treatments and controls. Animals not bearing tumors may also be subjected to the same treatments as the tumor-bearing animals in order to be able to dissociate the toxic effect from the specific effect on the tumor. Chemotherapy generally begins from 3 to 22 days after grafting, depending on the type of tumor, and the animals are observed every day. The different animal groups are weighed 3 or 4 times a week until the maximum weight loss is attained, and the groups are then weighed at least once a week until the end of the trial.

The tumors are measured 2 or 3 times a week until the tumor reaches approximately 2 g, or until the animal dies if this occurs before the tumor reaches 2 g. The animals are autopsied when sacrificed. The antitumour activity is determined in accordance with various recorded parameters.

The composition may be used without further modification by simply packaging it in vials and sterilizing it. A preferred sterilization method is to subject the composition to three sterilization cycles by autoclaving followed by incubation. The composition may also be used in a concentrated form. This solution is then tested for biological activity. The composition may also be lyophilized.

In other embodiments of the invention the compositions may be used in conjunction with known anticancer therapies or agents to enhance the effect of said therapies. In one specific embodiment the anticancer agent is a chemotherapeutic agent. One skilled in the art would appreciate how to test relative amounts of the compositions of the invention with anticancer agents (eg. chemotherapeutics) and/or anticancer therapies (eg. radiation-therapy).

Anticancer Agents

This invention provides for the use of BRM in combination with other anticancer compounds and antisense sequences. Examples of anti-cancer compounds are: antisense sequences; Bleomycin; Docetaxel (Taxotere); Doxorubicin; Edatrexate; Etoposide; Finasteride (Proscar); Flutamide (Eulexin); Gemcitabine (Gemzar); Goserelin Acetate (Zoladex); Granisetron (Kytril); Irinotecan (Campto/Camptosar); Leuprolide (Viadur); Methotrexate; Ondansetron (Zofran); Paclitaxel (Taxol); Pegaspargase (Oncaspar); Pilocarpine Hydrochloride (Salagen); Porfimer Sodium (Photofrin); Interleukin-2 (Proleukin); Rituximab (Rituxan); Topotecan (Hycamtin); Trastuzumab (Herceptin); Tretinoin (Retin-a); Triapine; Vincristine; Vinorelbine Tartrate (Navelbine); Drugs Used in Breast Cancer such as: Capecitabine (Xeloda); Cyclophosphamide (Cytoxan); Docetaxel (Taxotere); Doxorubicin Injection (Adriamycin); Doxorubicin, Liposomal-entrapped (Doxil); Epirubicin (Ellence); Exemestane (Aromasin); Raloxifene (Evista); Tamoxifen (Nolvadex); Trastuzumab (Herceptin); Goserelin Acetate (Zoladex), Zeneca; Drugs Used for Kaposi's Sarcoma such as: Liposomal-entrapped Doxorubicin (Doxil); Liposomal Daunorubicin (Daunoxome); Drugs for Promyelocytic Leukemia; Tretinoin (Vesanoid); Drugs for Chronic Myeloid Leukemia such as low-dose IFN-alpha; Drugs Used in Gastric Cancer; Antibiotics; Antineoplastics; Acute Lymphoblastic Leukemia; Pegaspargase (Oncaspar); L-asparaginase; IL-2; Drugs for Colon Cancer such as Edatrexate or 10-ethyl-10-deaza-aminopterin or 10edam; 5-fluorouracil (5-fu) and Levamisole; Methyl-ccnu (Methyl-chloroethyl-cyclohexyl-nitrosourea); Fluorodeoxyuridine (Fudr); Vincristine; Drugs for Esophageal Cancer such as: Porfimer Sodium (Photofrin) or Treatment with a Neodymium:yag (Nd:yag) Laser; Drugs Used in Colorectal Cancer such as: Irinotecan (Camptosat); Topotecan (Hycamtin); Loperamide (Imodium); 5-fluorouracil (5-fu); Drugs for Advanced Head and Neck Cancers such as Docetaxel (Taxotere); Drugs for Non-hodgkin's Lymphoma such as Rituximab; Etoposide; Drugs for Non-Small-Cell Lung Cancer such as a Vinca Alkaloid, Vinorelbine Tartrate (Navelbine): Paitaxel, (Taxol); Docetaxel (Taxotere); Topotecan; Irinotecan; Gemeitabine; Drugs for Ovarian Cancer: Docetaxel (Taxotere); Gemcitabine (Gemzar); Irinotecan (Camptosar); Paclitaxel (Taxol); Topotecan (Hycamtin); Amifostine (Ethyol), (For Reducing the Cumulative Renal Toxicity Associated with Repeated Cisplatin Therapy in Patients with Advanced Ovarian Cancer); Drugs to Prevent Melanoma such as: 2-ethylhexyl-p-methoxy-cinnamate (2-ehmc); Octyl-N-dimethyl-p-aminobenzoate (O-paba); Benzophenone-3 (Bp3); Drugs for Prostate Cancer such as: Flutamide (Eulexin); Finasteride (Proscar); Terazosin (Hytrin); Doxazosin (Cardura); Goserelin Acetate (Zoladex); Liarozole; Nilutamide (Nilandron); Mitoxantrone (Novantrone); Prednisone (Deltasone); Drugs for Pancreatic Cancer such as Gemcitabine (Gemzar); 5-fluorouracil; Drugs for Advanced Renal Cancer such as Interleukin-2 (Proleukin); Other Anti-neoplastic Drugs such as Porfimer Sodium, Axcan; Dacarbazine; Etoposide; Procarbazine HCl; Rituximab; Paclitaxel (Taxol), Trastuzumab (Herceptin); Temozolomide (Temodal); Alkylating Agents Used in Combination Therapy for Different Cancers such as Cyclophosphamide; Cisplatin; Melphalan; Therapies used for treatment of Cancer such as Photodynamics and photosensitizing agents; Surgery; Cryotherapy; Chemotherapy; Biotherapy; immunotherapy (similar to biotherapy); Angiogenesis inhibitors; and hormone replacement therapy.

Antisense Compounds

The specificity and sensitivity of antisense compounds makes them useful in diagnostics, therapeutics, prophylaxis, as research reagents and in kits. In the context of the present invention, the terms “antisense compound” and “antisense oligonucleotide” each refer to an oligomer or polymer of ribonucleic acid (RNA), or deoxyribonucleic acid (DNA), or mimetics thereof. These terms also include chimeric antisense compounds, which are antisense compounds that contain two or more chemically distinct regions, each made up of at least one monomer unit. In accordance with the present invention, the terms “antisense compound” and “antisense oligonucleotide” further include oligonucleotides composed of naturally occurring nucleobases, sugars and covalent internucleoside (backbone) linkages, as well as oligonucleotides comprising non-naturally-occurring moieties that function similarly. Such modified or substituted oligonucleotides are well known to workers skilled in the art and often preferred over native forms because of desirable properties such as, for example, enhanced cellular uptake, enhanced affinity for nucleic acid target and increased stability in the presence of nucleases. The antisense compounds in accordance with the present invention comprise from about 7 to about 50 nucleobases, or from about 7 to about 30. Alternatively, the antisense compounds comprise a mixture of short oligomers which will bind to the target nucleic acid in tandem (i.e. they are complementary to sequences that are adjacent to one another in the target nucleic acid).

Examples of antisense compounds useful in the present invention include oligonucleotides containing modified backbones or non-natural internucleoside linkages. In accordance with the present invention, oligonucleotides having modified backbones include those that retain a phosphorus atom in the backbone and those that do not have a phosphorus atom in the backbone. For the purposes of the present invention, and as sometimes referenced in the art, modified oligonucleotides that do not have a phosphorus atom in their internucleoside backbone can also be considered to be oligonucleosides.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may be additionally or alternatively employed. Similar techniques using phosphorothioates and alkylated derivatives have been employed to produce oligonucleotides.

Antisense oligonucleotides have been successfully employed as therapeutic moieties in the treatment of disease states such as cancer. Antisense compounds exert their effects by specifically modulating expression of a gene implicated in a specific disease state. Thus, the present invention contemplates the therapeutic administration of an effective amount of a combination of the Syn-BRM composition of the present invention and an appropriate antisense compound to a mammal suspected of having a disease or disorder which can be treated by specifically modulating gene expression. The present invention further contemplates the prophylactic use of a combination of the Syn-BRM composition and an antisense compound in the prevention of a cancer which is related to over- or under-expression of a specific gene.

Pharmaceutical Compositions

The compositions of the invention may be converted using customary methods into pharmaceutical compositions. The pharmaceutical composition containing the composition of the invention either alone or together with other active substances. Such pharmaceutical compositions can be for oral, topical, rectal, parenteral, local, inhalant, or intracerebral use. They are therefore in solid or semisolid form, for example pills, tablets, creams, gelatin capsules, capsules, suppositories, soft gelatin capsules, gels, membranes, and tubelets. For parenteral and intracerebral uses, those forms for intramuscular or subcutaneous administration can be used, or forms for infusion or intravenous or intracerebral injection can be used, and can therefore be prepared as solutions of the compositions or as powders of the active compositions to be mixed with one or more pharmaceutically acceptable excipients or diluents, suitable for the aforesaid uses and with an osmolarity that is compatible with the physiological fluids. For local use, those preparations in the form of creams or ointments for topical use or in the form of sprays may be considered; for inhalant uses, preparations in the form of sprays, for example nose sprays, may be considered. Preferably, the composition is administered intramuscularly.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Nack Publishing Company, Easton, Pa, USA 1985).

On this basis, the pharmaceutical compositions include, albeit not exclusively, the composition of the invention in association with one or more pharmaceutically acceptable vehicles or diluents, and are contained in buffered solutions with a suitable pH and iso-osmotic with the physiological fluids.

The compositions are indicated as therapeutic agents either alone or in conjunction with other therapeutic agents or other forms of treatment. For example, other antiviral compounds, including but not limited to; 3TC, interferon, ganciclovir, famciclovir, rimantadine, foscarnet sodium, zidovudine, amantadine hydrochloride, valacyclovir, ribavirin, acyclovir, may be used in combination with the composition of the present invention. The compositions and agents of the invention are intended for administration to humans or animals.

It will be appreciated by medical practitioners that it may be necessary to deviate from the amounts recommended and, in particular, to do so as a function of the body weight and condition of the mammal to be treated, the particular disease to be treated, the nature of the administration route and the therapy desired. In addition, the type of mammal and its individual behavior towards the medicine or the nature of its formulation and the time or interval at which it is administered may also indicate use of amounts different from those mentioned. Thus it may suffice, in some cases, to manage with less than the above-mentioned minimum amounts while in other cases the upper limit mentioned must be exceeded. Where major amounts are administered, it may be advisable to divide these into several administrations over the course of the day.

The present invention comprises a composition and it's use either alone or in combination with other drugs or therapies, wherein the composition shows no cytotoxicity to human peripheral blood mononuclear cells, and has at least one of the following properties:

(a) is capable of stimulating monocytes and/or macrophages in vitro or in vivo to produce one or more cytokines; and/or

(b) is capable of stimulating monocytes and/or macrophages to produce tumor necrosis factor in vitro and/or in vivo;

In a preferred embodiment of the composition, the composition stimulates tumor necrosis factor production in vitro or in vivo, and most preferably in humans.

Therapeutic Activity of the Combination

The combinations of the present invention will have a net anticancer effect that is greater than the anticancer effect of the individual components of the combination when administered alone. Without being limited by mechanism, by combining one or more anticancer agents with a Syn-BRM composition it is possible to:

(i) increase the therapeutic effect of the anticancer agent(s);

(ii) increase the therapeutic effect of the Syn-BRM composition;

(iii) decrease or delay the toxicity phenomena associated with the anticancer agent(s); and/or

(iv) decrease or delay the toxicity phenomena associated with the Syn-BRM composition,

in comparison to treatment with the individual components of the combination.

In one embodiment the combination of the present invention provides an improved efficacy, over treatment using the components of the combination alone, that may be demonstrated by determination of the therapeutic synergy.

A combination manifests therapeutic synergy if it is therapeutically superior to one or other of the constituents used at its optimum dose [T. H. Corbett et al., (1982) Cancer Treatment Reports, 66, 1187]. To demonstrate the efficacy of a combination, it may be necessary to compare the maximum tolerated dose of the combination with the maximum tolerated dose of each of the separate constituents in the study in question. This efficacy may be quantified using techniques and equations commonly known to workers skilled in the art. [T. H. Corbett et al., (1977) Cancer, 40, 2660.2680; F. M. Schabel et al., (1979) Cancer Drug Development, Part B, Methods in Cancer Research, 17, 3-51, New York, Academic Press Inc.].

The combination, used at its own maximum tolerated dose, in which each of the constituents will be present at a dose generally not exceeding its maximum tolerated dose, will manifest therapeutic synergy when the efficacy of the combination is greater than the efficacy of the best constituent when it is administered alone.

In another embodiment the combination of the present invention improves the therapeutic index in the treatment of cancer over that of the Syn-BRM composition or the anticancer agent(s) when administered to a patient alone.

A median effective dose (ED₅₀) of a drug is the dose required to produce a specified effect in 50% of the population. Similarly, the median lethal dose (LD₅₀) of a drug, as determined in preclinical studies, is the dose that has a lethal effect on 50% of experimental animals. The ratio of the LD₅₀ to the ED₅₀ can be used as an indication of the therapeutic index. Alternatively the therapeutic index can be determined based on doses that produce a therapeutic effect and doses that produce a toxic effect for other proportions of the treated population. Examples include, but are not limited to EDₓ, where x=90, 95 or 99 and LD_{y}, where y=10, 5 or 1 respectively (note that the values of x and y need not add up to 100). It is well known in the art that the acceptability of therapeutic index value (for any given EDₓ/LD_{y} ratio) varies depending on the severity of the disease and availability of other more efficacious and/or less toxic treatment options. During clinical studies, the dose, or the concentration (e.g. solution, blood, serum, plasma), of a drug required to produce toxic effects can be compared to the concentration required for the therapeutic effects in the population to evaluate the clinical therapeutic index. Methods of clinical studies to evaluate a clinical therapeutic index are well known to workers skilled in the art.

In one embodiment the combination of the present invention provides an improved therapeutic index, in comparison to that of the individual components of the combination when administered alone, by decreasing, for example, the observed LDy of at least one of the one or more anticancer agents in the combination.

In a related embodiment the combination of the present invention provides an improved therapeutic index, in comparison to that of the individual components of the combination when administered alone, by increasing the observed EDₓ of at least one of the one or more anticancer agents in the combination. In a further embodiment the combination of the present invention provides an improved therapeutic index, in comparison to that of the individual components of the combination when administered alone, by increasing the observed EDₓ of the Syn-BRM.

In another embodiment the efficacy of a combination according to the present invention may also be characterized by adding the actions of each constituent.

In order to prepare a combination according to the present invention one first selects one or more candidate anticancer agent(s) and measure its efficacy in a model of a cancer of interest, as would be well understood by one skilled in the art. The next step may be to perform a routine analysis to compare the efficacy of the one or more anticancer agent(s) alone to the efficacy of the one or more anticancer agent(s) in combination with varying amounts of the Syn-BRM composition. Successful candidates for use in the combinations of the present invention will be those that demonstrate a therapeutic synergy with the Syn-BRM or that improve the therapeutic index in comparison to the therapeutic index of the candidate agent(s).

The efficacy of the combinations of the present invention may be determined experimentally using standard techniques using cancer models well known to workers skilled in the art. Such cancer models allow the activity of combinations to be tested in vitro and in vivo in relation to the cancer of interest. Exemplary methods of testing-activity are described in the Examples provided herein, although, it should be understood that these methods are not intended to limit the present invention.

One example of a method for studying the efficacy of the combinations on solid tumors in vivo involves the use of subject animals, generally mice, that are subcutaneously grafted bilaterally with 30 to 60 mg of a tumor fragment on day 0. The animals bearing tumors are mixed before being subjected to the various treatments and controls. In the case of treatment of advanced tumors, tumors are allowed to develop to the desired size, animals having insufficiently developed tumors being eliminated. The selected animals are distributed at random to undergo the treatments and controls. Animals not bearing tumors may also be subjected to the same treatments as the tumor-bearing animals in order to be able to dissociate the toxic effect from the specific effect on the tumor. Chemotherapy generally begins from 3 to 22 days after grafting, depending on the type of tumor, and the animals are observed every day. The different animal groups are weighed 3 or 4 times a week until the maximum weight loss is attained, and the groups are then weighed at least once a week until the end of the trial.

The tumors are measured 2 or 3 times a week until the tumor reaches approximately 2 g, or until the animal dies if this occurs before the tumor reaches 2 g. The animals are autopsied when sacrificed. The antitumour activity is determined in accordance with various recorded parameters.

For a study of the combinations on leukaemias, the animals are grafted with a particular number of cells, and the antitumour activity is determined by the increase in the survival time of the treated mice relative to the controls.

Administration of the Combination

The uses and methods of the present invention comprise administering to a subject in need thereof an effective amount of a Syn-BRM composition in combination with one or more anticancer agents to a subject. As used herein, combination components are said to be administered in combination when the two or more components are administered simultaneously or are administered independently in a fashion such that the components will act at the same time.

Components administered independently can, for example, be administered separately (in time) or concurrently. Separately in time means at least minutes apart, and potentially hours, days or weeks apart. The period of time elapsing between the administration of the components of the combination of the invention can be determined by a worker of skill in the art, and will be dependent upon, for example, the age, health, and weight of the recipient, nature of the combination treatment, side effects associated with the administration of other component(s) of the combination, frequency of administration(s), and the nature of the effect desired. Components of the combinations of the invention may also be administered independently with respect to location and, where applicable, route of administration.

In another embodiment, an effective amount of a therapeutic composition comprising a Syn-BRM composition and one or more anticancer agents, and a pharmaceutically acceptable carrier is administered to a subject. The combination or the pharmaceutical composition of the invention can be administered before during or after other anticancer treatment(s), or treatments for other diseases or conditions. For example a drug to treat adverse side effects of the anticancer treatment(s) can be administered concurrently with a combination of the invention or a pharmaceutical composition of the invention.

As indicated above the components of the combination of the present invention may be administered separately, concurrently, or simultaneously. In the case of separate administration the Syn-BRM composition may be administered before, during or after administration of the anticancer agent(s). Furthermore, it would be readily apparent to a worker skilled in the art that the route of administration of each component of the combination is selected in order to maximize the therapeutic benefit of the component and it is not necessary that each component be delivered via the same route. The Syn-BRM composition and/or the anticancer agent(s) of the combination may be administered via a single dose or via continuous perfusion.

The agents, compounds and compositions of this invention can be utilised in vivo, ordinarily in mammals, such as humans, sheep, horses, cattle, pigs, dogs, cats, rats and mice, or in vitro to treat cancer or cancer cells.

Cancers

As used herein, “cancer” refers to all types of cancer or neoplasm or malignant tumors found in mammals, including carcinomas and sarcomas. Examples of cancers are cancer of the brain, breast, cervix, colon, head and neck, kidney, lung, non-small cell lung, melanoma, mesothelioma, ovary, sarcoma, stomach, uterus and Medulloblastoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia

The term “sarcoma” generally refers to a tumor which is made up of a substance like the embryonic connective tissue and is generally composed of closely packed cells embedded in a fibrillar or homogeneous substance. Sarcomas include chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma viflosum.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

Pharmaceutical Kits

The present invention additionally provides for therapeutic kits containing (i) a dosage unit of a composition and a pharmaceutically acceptable carrier; and (ii) dosage unit of one or more chemotherapeutic drug(s) and a pharmaceutically acceptable carrier, said (i) and (ii) being provided in amounts that are effective, in combination, for selectively killing tumor or metastatic cells.

As used herein, a “dosage unit” is a pharmaceutical composition or formulation comprising at least one active ingredient and optionally one or more inactive ingredient(s). The dosage unit can be unitary, such as a single pill or liquid, containing all of the desired active ingredients and the inactive ingredients necessary and desired for making a dosage suitable for administration (e.g., tabletting compounds such as binders, fillers, and the like); the dosage unit can consist of a number of different dosage forms (e.g., pill(s) and/or liquid(s)) designed to be taken simultaneously as a dosage unit.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

### EXAMPLES

### Example 1

### Synthetic BRM Compositions

A synthetic BRM composition was prepared by combining the compounds in the approximate amounts as shown in Table 1. This composition is referred to herein as Synthetic BRM #1.

A synthetic BRM composition was prepared by combining the compounds listed in Table 2 in the approximate amounts as indicated. These compositions are referred to herein as Synthetic BRM #2.

A Syn-BRM composition was prepared according to Table 3b (Synthetic BRM #4)

The above compositions are biological response modifier compositions each comprising the components: 3-hydroxybutyric acid, lactic acid, acetic acid, creatine, creatinine, carnitine, taurine, choline, urea. Synthetic BRM #4 additionally comprises formic acid.

These Syn-BRM compositions are shown to have activity similar to Nat-BRM. The compositions are compared to naturally occurring BRM compositions, isolated from animal bile, referred to herein as Nat-BRM. These are also described using batch numbers such as # 311 or #313, or alternatively as BD-BRM.

### Example 2

### In Vivo Demonstration of Efficacy of SYN-BRM Composition in the Treatment of Human Pancreatic Carcinoma in CD-1 Nude Mice

This experiment demonstrates and compares the ability of two synthetic BRM compositions: Syn-BRM#1 and Syn-BRM#1, to inhibit the growth of human pancreatic carcinoma (BxPC-3) in CD-1 nude mice.

Human pancreatic carcinoma cell line (BxPC-3) were grown as monolayer culture in Minimum essential medium (α-MEM) supplemented with 10% fetal bovine serum (FBS), 0.1 mM non-essential amino acid, 1.0 mM sodium pyruvate, 100 U/ml penicillin, 100 μg/ml streptomycin, 0.25 μg/ml amphotericin B and 2 mM L-alanyl-1-glutamine at 37° C. in an atmosphere of 5% CO₂ in air. The tumor cells were routinely subcultured twice weekly by trypsin-EDTA treatment. The cells were harvested from subconfluent logarithmically growing culture by treatment with trypsin-EDTA and counted for tumor inoculation.

An acclimation period of at least 7 days was allowed between animal receipt and commencement of tumor inoculation. When the female CD-1 mice were 6-7 weeks of age (20-25 g), each mouse was subcutaneously injected at the right flank with 3×10⁶ BxPC-3 human pancreatic carcinoma cells in 0.1 ml of PBS to induce tumor growth.

The following treatment (or control) conditions were evaluated for this demonstration.

Group 1: Saline Control (0.2 ml/mouse/day, i.p., n=10)

Group 2: Syn-BRM #1 (0.2 ml/mouse/day, i.p., n=10)

Group 3: Syn-BRM #2 (0.2 ml/mouse/day, i.p., n=10)

The major endpoint was to demonstrate that tumor growth could be delayed or mice could be cured by the treatment with synthetic BRM compositions and to demonstrate how the anti-tumor effects of different BRM compositions compare with each other. Tumor sizes were measured every other day from day 19 after the tumor cell inoculation in two dimensions using a caliper, and the volume was expressed in mm³ using the formula: V=0.5 a×b², where a and b are the long and short diameters of the tumor, respectively. The treatments were terminated at day 68. Mean tumor volumes calculated from each measurement were then plotted in a standard graph to compare the anti-tumor efficacy of drug treatments to that of control. One day after the last treatment, tumors were excised from the animals and tumor weights were measured. A standard bar graph was used to demonstrate the differences in tumor weights with each bar representing mean tumor weight calculated from 10 animals.

The purpose of this study was to demonstrate efficacy of Syn-BRM compositions based on the components. Two embodiments of synthetic BRM=s showed biological responses that were similar to the natural BRM (Nat-BRM) with respect to TNF-α release. The in vivo nude mouse xenograft model of BxPC-3 human pancreatic carcinoma has been tested using these two synthetic BRM compositions.

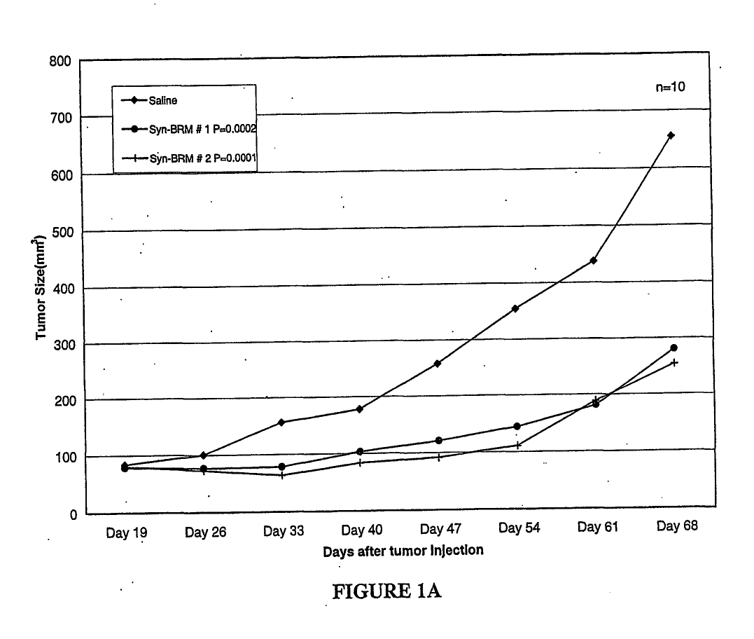

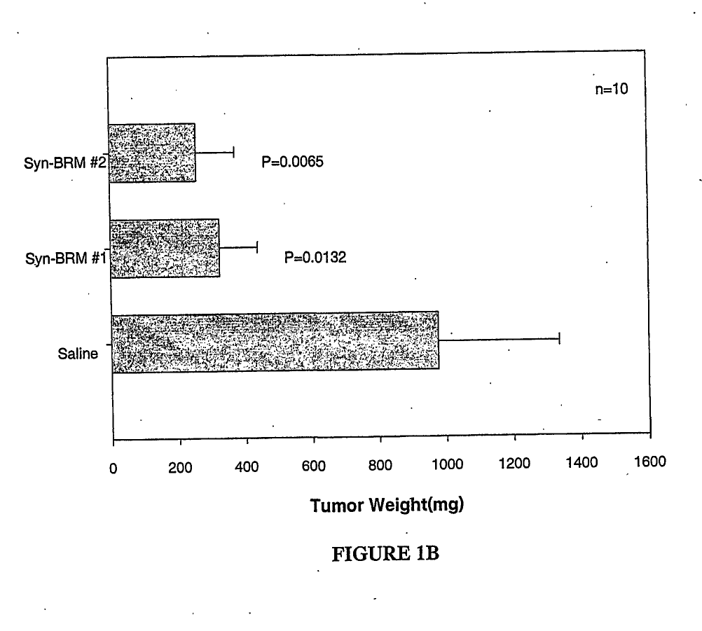

The results of the tumor growth curve and tumor weight measurements are shown in the FIGS. 1(A) and (B). As illustrated, treatment with each of the two synthetic BRM compositions resulted in significant delay of tumor growth compared to saline control.

### Example 3

### In Vivo Demonstration of Efficacy of SYN-BRM in the Treatment of Human Pancreatic Carcinoma in CD-1 Nude Mice

This experiment demonstrates and compares the ability of two synthetic BRM compositions and a natural BRM (batch #311) to inhibit the growth of human pancreatic carcinoma (BxPC-3) in CD-1 nude mice.

Human pancreatic carcinoma cell line (BxPC-3) was grown as monolayer culture in Minimum essential medium (α-MEM) supplemented with 10% fetal bovine serum (FBS), 0.1 mM non-essential amino acid, 1.0 mM sodium pyruvate, 100 U/ml penicillin, 100 μg/ml streptomycin, 0.25 μg/ml amphotericin B and 2 mM L-alanyl-1-glutamine at 37° C. in an atmosphere of 5% CO₂ in air. The tumor cells were routinely subcultured twice weekly by trypsin-EDTA treatment. The cells were harvested from subconfluent logarithmically growing culture by treatment with trypsin-EDTA and counted for tumor inoculation.

An acclimation period of at least 7 days was allowed between animal receipt and commencement of tumor inoculation. When the female CD-1 mice were 6-7 weeks of age (20-25 g), each mouse was subcutaneously injected at the right flank with 3×10⁶ BxPC-3 human pancreatic carcinoma cells in 0.1 ml of PBS to induce tumor growth.

The following treatment (or control) conditions were evaluated for this experiment.

Group 1: Saline Control (0.2 ml/mouse/day, i.p., n—10)

Group 2: Natural BRM (batch #311) (0.2 ml/mouse/day, i.p., n=10)

Group 3: Synthetic BRM #1. (0.2 ml/mouse/day, i.p., n=10)

Group 4: Synthetic BRM #2 (0.2 ml/mouse/day, i.p., n=10)

The major endpoint was to demonstrate that tumor growth could be delayed or mice could be cured by the treatment with natural BRM or synthetic BRM compositions and to see how the anti-tumor effects of different synthetic BRM compositions compare with those of natural BRM. Tumor sizes were measured every other day from day 23 after the tumor cell inoculation in two dimensions using a caliper, and the volume was expressed in mm³ using the formula: V=0.5 a×b², where a and b are the long and short diameters of the tumor, respectively. The treatments were terminated at day 58. Mean tumor volumes calculated from each measurement were then plotted in a standard graph to compare the anti-tumor efficacy of drug treatments to that of control. One day after the last treatment, tumors were excised from the animals and tumor weights were measured. A standard bar graph was used to demonstrate the differences in tumor weights with each bar representing mean tumor weight calculated from 10 animals.

The purpose of this study was to demonstrate the efficacy of synthetic BRM based on the components in Table 1. Two embodiments of a synthetic BRM used in this study showed biological responses that were similar to the natural BRM with respect to TNF-α release. The in vivo nude mouse xenograft model of BxPC-3 pancreatic carcinoma has been tested using these two synthetic BRM compositions and their antitumor efficacy has been compared with that of natural BRM.

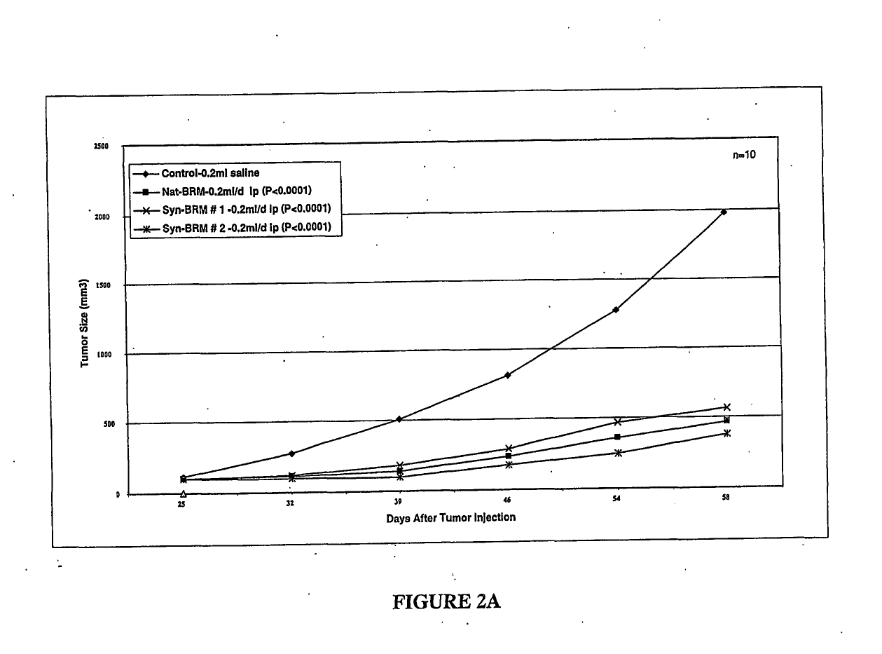

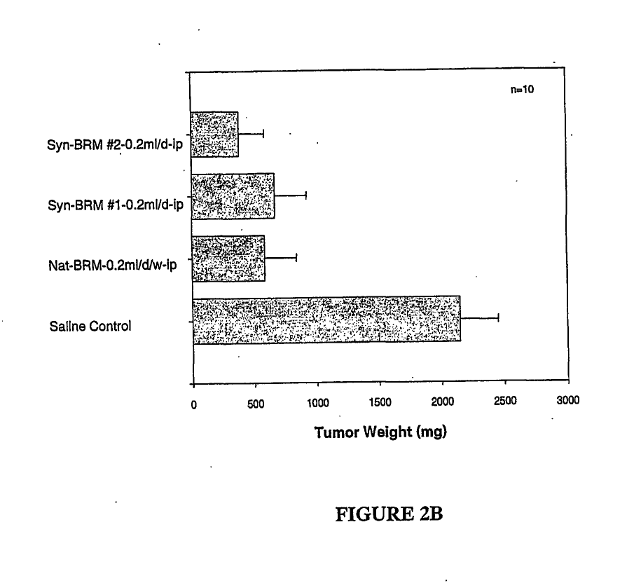

The results of the tumor growth curve and tumor weight measurements-are shown in FIG. 2 (A) and (B). As illustrated, treatment with each of the two synthetic BRM compositions resulted in significant delay of tumor growth compared to saline control. The delay in tumor growth achieved with both synthetic BRM compositions was as effective as that observed with the natural BRM.

### Example 4

### In Vivo Demonstration of Efficacy of Synthetic BRM in the Treatment of Human Melanoma in CD-1 Nude Mice

This experiment demonstrates and compares the ability of two embodiments of a synthetic BRM composition and a natural BRM (batch #311), to inhibit the growth of human melanoma (C8161) in CD-1 nude mice.

Human melanoma cell line (C8161) was grown as monolayer culture in Minimum essential medium (α-MEM) supplemented with 10% fetal bovine serum (FBS), 0.1 mM non-essential amino acid, 1.0 mM sodium pyruvate, 100 U/ml penicillin, 100 μg/ml streptomycin, 0.25 μg/ml amphotericin B and 2 mM L-alanyl-1-glutamine at 37° C. in an atmosphere of 5% CO₂ in air. The tumor cells were routinely subcultured twice weekly by trypsin-EDTA treatment. The cells were harvested from subconfluent logarithmically growing culture by treatment with trypsin-EDTA and counted for tumor inoculation.

An acclimation period of at least 7 days was allowed between animal receipt and commencement of tumor inoculation. When the female CD-1 mice were 6-7 weeks of age (20-25 g), each mouse was subcutaneously injected with 5×10⁶ C8161 human melanoma cells in 0.1 ml of PBS at the right flank of the mice to induce tumor growth.

The following treatment (or control) conditions were evaluated for this experiment.

Group 1: Saline Control (0.2 ml/mouse/day, i.p., n110)

Group 2: Natural BRM (batch #311) (0.2 ml/mouse/day, i.p., n=10)

Group 3: Synthetic BRM #1 (0.2 ml/mouse/day, i.p., n=10)

Group 4: Synthetic BRM #2 (0.2 ml/mouse/day, i.p., n=10)

The major endpoint was to demonstrate that tumor growth could be delayed or mice could be cured by the treatment with either the natural BRM or the synthetic BRM compositions and to see how the anti-tumor effects of different synthetic BRM compositions compare with those of natural BRM. Tumor sizes were measured every other day from day 5 after the tumor cell inoculation in two dimensions using a caliper, and the volume was expressed in mm³ using the formula: V=0.5 a×b², where a and b are the long and short diameters of the tumor, respectively. The treatments were terminated at day 38. Mean tumor volumes calculated from each measurement were then plotted in a standard graph to compare the anti-tumor efficacy of drug treatments to that of control. One day after the last treatment, tumors were excised from the animals and tumor weights were measured. A standard bar graph was used to demonstrate the differences in tumor weights with each bar representing mean tumor weight calculated from 10 animals.

The purpose of this study was to demonstrate the efficacy of a synthetic BRM based on the components presented in Table 1. Two embodiments of a synthetic BRM were used in this study showed biological responses that were similar to the natural BRM with respect to TNF-α release. The in vivo nude mouse xenograft model of C8161 melanoma has been tested using these two synthetic BRM compositions and their antitumor efficacy has been compared with that of natural BRM.

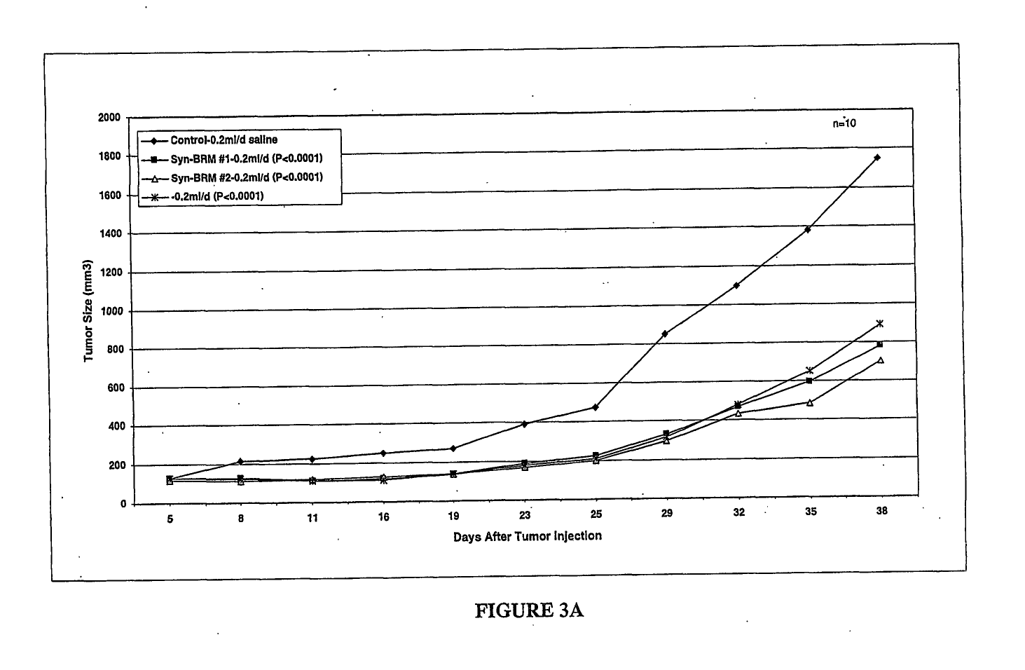

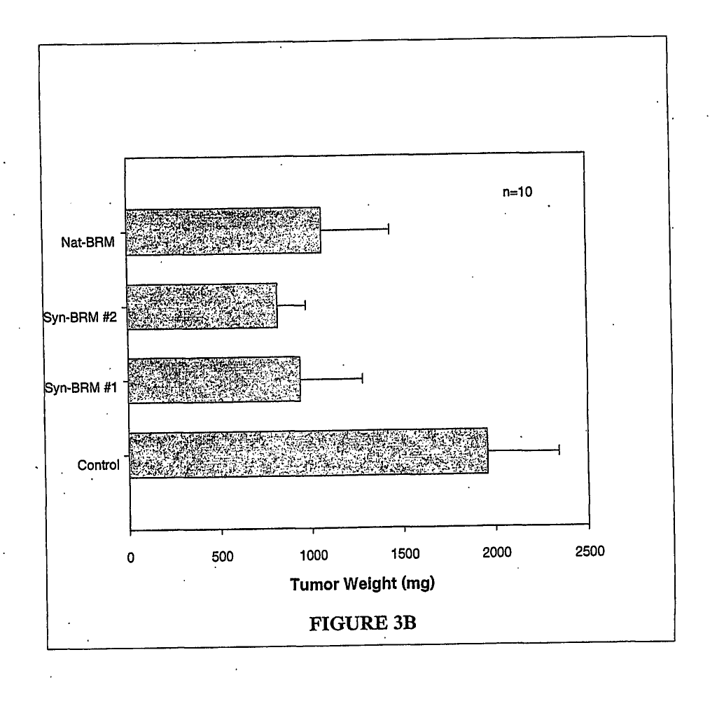

The results of the tumor growth curve and tumor weight measurements are shown in FIGS. 3 (A) and (B). As illustrated, treatment with each of the two synthetic BRM compositions resulted in significant delay of tumor growth compared to saline control. The delay in tumor growth achieved with synthetic BRM #1 and #2 compositions was as effective as that observed with natural BRM.

### Example 5

### In Vivo Demonstration of Efficacy of Synthetic BRM in the Treatment of Human Breast Adenocarcinoma in CD-1 Nude Mice

This experiment demonstrates and compares the ability of two synthetic BRM compositions and a natural BRM (batch #311), to inhibit the growth of human breast adenocarcinoma (MDA-MB-231) in CD-1 nude mice.

Human breast adenocarcinoma cell line (MDA-MB-231) was grown as monolayer culture in Minimum essential medium (α-MEM) supplemented with 10% fetal bovine serum (FBS), 0.1 mM non-essential amino acid, 1.0 mM sodium pyruvate, 100 U/ml penicillin, 100 μg/ml streptomycin, 0.25 μg/ml amphotericin B and 2 mM L-alanyl-1-glutamine at 37° C. in an atmosphere of 5% CO₂ in air. The tumor cells were routinely subcultured twice weekly by trypsin-EDTA treatment. The cells were harvested from subconfluent logarithmically growing culture by treatment with trypsin-EDTA and counted for tumor inoculation.

An acclimation period of at least 7 days was allowed between animal receipt and commencement of tumor inoculation. When the female CD-1 mice were 6-7 weeks of age (20-25 g), each mouse was subcutaneously injected at the right flank with 1×10⁷ MDA-MB-231 human breast adenocarcinoma cells in 0.1 ml of PBS to induce tumor growth.

The following treatment (or control) conditions were evaluated for this experiment.

Group 1: Saline Control (0.2 ml/mouse/day, i.p., n=8)

Group 2: Natural BRM (batch #311) (0.2 ml/mouse/day, i.p., n=8)

Group 3: Synthetic BRM #1 (0.2 ml/mouse/day, i.p., n=8)

Group 4: Synthetic BRM #2 (0.2 ml/mouse/day, i.p., n=8)

The major endpoint was to demonstrate that tumor growth could be delayed or mice could be cured by the treatment with either the natural BRM or the synthetic BRM compositions and to see how the anti-tumor effects of different synthetic BRM compositions compare with those of natural BRM. Tumor sizes were measured every other day from day 4 after the tumor cell inoculation in two dimensions using a caliper, and the volume was expressed in mm³ using the formula: V=0.5 a×b², where a and b are the long and short diameters of the tumor, respectively. The treatments were terminated at day 29. Mean tumor volumes calculated from each measurement were then plotted in a standard graph to compare the anti-tumor efficacy of drug treatments to that of control. One day after the last treatment, tumors were excised from the animals and tumor weights were measured. A standard bar graph was used to demonstrate the differences in tumor weights with each bar representing mean tumor weight calculated from 8 animals.

The purpose of this study was to demonstrate the efficacy of a synthetic BRM based on the components presented in Table 1. Two embodiments of a synthetic BRM used in this study showed biological responses that were similar to the natural BRM with respect to TNF-α release. The in vivo nude mouse xenograft model of MDA-MB-231 breast adenocarcinoma has been tested using these two synthetic BRM compositions and their antitumor efficacy has been compared with that of natural BRM.

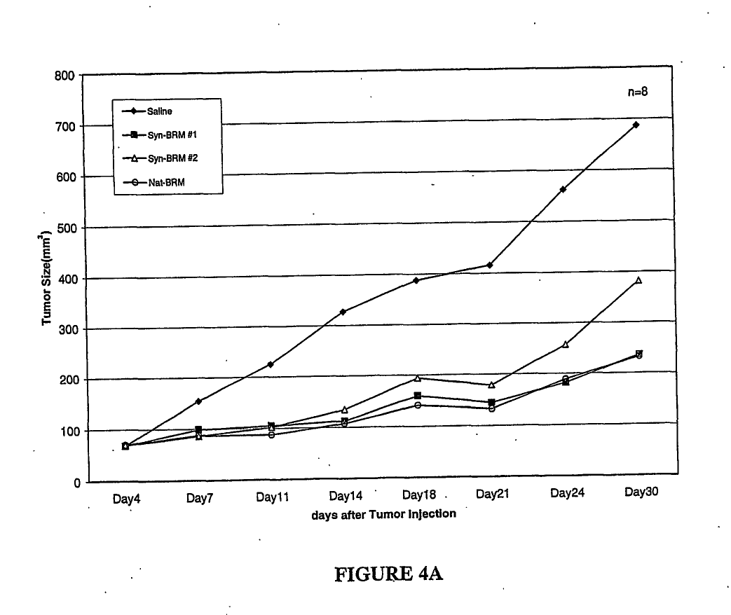

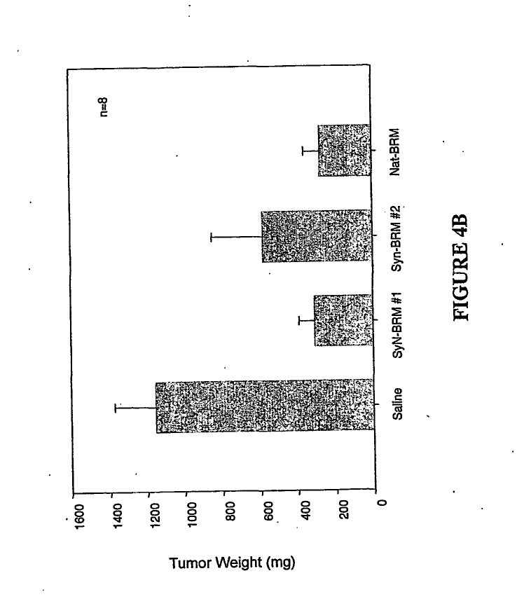

The results of the tumor growth curve and tumor weight measurements are shown in FIGS. 4 (A) and (B). As illustrated, treatment with each of the two synthetic BRM compositions resulted in significant delay of tumor growth compared to saline control. The delay in tumor growth achieved with synthetic BRM #1 was as effective as that observed with natural BRM.

### Example 6

### In Vivo Demonstration of Efficacy of a Synthetic BRM Composition in the Treatment of Human Pancreatic Carcinoma in CD-1 Nude Mice

This experiment demonstrates and compares the ability of two embodiments of a synthetic BRM composition and a natural BRM (batch #313), to inhibit the growth of human pancreatic carcinoma (BxPC-3) in CD-1 nude mice.

Human pancreatic carcinoma cell line (BxPC-3) was grown as monolayer culture in Minimum essential medium (α-MEM) supplemented with 10% fetal bovine serum (FBS), 0.1 mM non-essential amino acid, 1.0 mM sodium pyruvate, 100 U/ml penicillin, 100 μg/ml streptomycin, 0.25 μg/ml amphotericin B and 2 mM L-alanyl-1-glutamine at 37° C. in an atmosphere of 5% CO₂ in air. The tumor cells were routinely subcultured twice weekly by trypsin-EDTA treatment. The cells were harvested from subconfluent logarithmically growing culture by treatment with trypsin-EDTA and counted for tumor inoculation.

An acclimation period of at least 7 days was allowed between animal receipt and commencement of tumor inoculation. When the female CD-1 mice were 6-7 weeks of age (20-25 g), each mouse was subcutaneously injected at the right flank with 3×10⁶ BxPC-3 human pancreatic carcinoma cells in 0.1 ml of PBS to induce tumor growth

The following treatment (or control) conditions were evaluated for this experiment

Group 1: Saline Control (0.2 ml/mouse/day, i.p., n=10)

Group 2: Natural BRM (batch #313) (0.2 ml/mouse/day, i.p., n=10)

Group 3: Synthetic BRM #1 (0.2 ml/mouse/day, i.p., n=10)

Group 4: Synthetic BRM #2 (0.2 ml/mouse/day, i.p., n=10)

The major endpoint was to demonstrate that tumor growth could be delayed or mice could be cured by the treatment with natural BRM or synthetic BRM compositions and to demonstrate how the anti-tumor effects of different synthetic BRM compositions compare with those of natural BRM. Tumor sizes were measured every other day from day 17 after the tumor cell inoculation in two dimensions using a caliper, and the volume was expressed in mm³ using the formula: V=0.5 a×b², where a and b are the long and short diameters of the tumor, respectively. The treatments were terminated at day 64. Mean tumor volumes calculated from each measurement were then plotted in a standard graph to compare the anti-tumor efficacy of drug treatments to that of control. One day after the last treatment, tumors were excised from the animals and tumor weights were measured. A standard bar graph was used to demonstrate the differences in tumor weights with each bar representing mean tumor weight calculated from 10 animals.

The purpose of this study was to demonstrate the efficacy of a synthetic BRM based on the components in Table 1. Two embodiments of a synthetic BRM composition used in this study showed biological responses that were similar to the natural BRM with respect to TNF-α release. The in vivo nude mouse xenograft model of BxPC-3 pancreatic carcinoma has been tested using these two embodiments of a synthetic BRM composition and their antitumor efficacy has been compared with that of natural BRM.

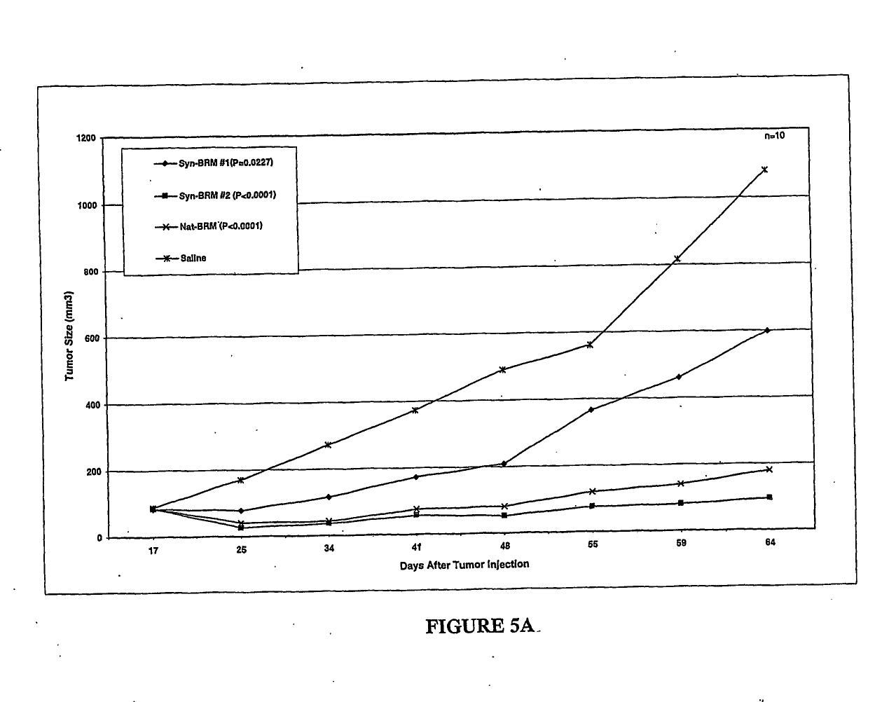

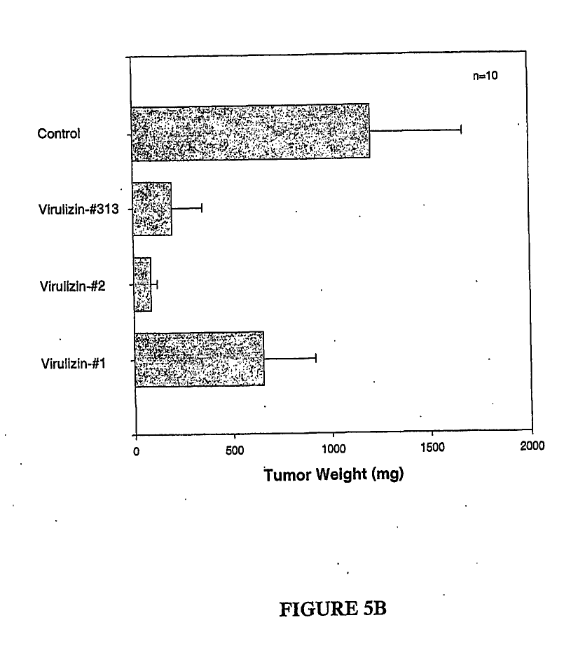

The results of the tumor growth curve and tumor weight measurements are shown in FIGS. 5(A) and (B). As illustrated, treatment with each of the two synthetic BRM compositions resulted in significant delay of tumor growth compared to saline control. The delay in tumor growth achieved with synthetic BRM #2 was as effective as that observed with the natural BRM.

### Example 7

### In Vivo Demonstration of Efficacy of a Synthetic BRM Composition in the Treatment of Human Breast Adenocarcinoma in CD-1 Nude Mice

This experiment demonstrates and compares the ability of two embodiments of a synthetic BRM composition and a natural BRM (batch #313), to inhibit the growth of human breast adenocarcinoma (MDA-MB-231) in CD-1 nude mice.

Human breast adenocarcinoma cell line (MDA-MB-231) was grown as monolayer culture in Minimum essential medium (α-MEM) supplemented with 10% fetal bovine serum (FBS), 0.1 mM non-essential amino acid, 1.0 mM sodium pyruvate, 100 U/ml penicillin, 100 μg/ml streptomycin, 0.25 μg/ml amphotericin B and 2 mM L-alanyl-1-glutamine at 37° C. in an atmosphere of 5% CO₂ in air. The tumor cells were routinely subcultured twice weekly by trypsin-EDTA treatment. The cells were harvested from subconfluent logarithmically growing culture by treatment with trypsin-EDTA and counted for tumor inoculation.

An acclimation period of at least 7 days was allowed between animal receipt and commencement of tumor inoculation. When the female CD-1 mice were 6-7 weeks of age (20-25 g), each mouse was subcutaneously injected at the right flank with 1×10⁷ MDA-MB-231 human breast adenocarcinoma cells in 0.1 ml of PBS to induce tumor growth.

The following treatment (or control) conditions were evaluated for this experiment.

Group 1: Saline Control (0.2 ml/mouse/day, i.p., n=10)

Group 2: Natural BRM (batch #313) (0.2 ml/mouse/day, i.p., n=10)

Group 3: Synthetic BRM #1 (0.2 ml/mouse/day, i.p., n=10)

Group 4: Synthetic BRM #2 (0.2 ml/mouse/day, i.p., n=10)

The major endpoint was to demonstrate that tumor growth could be delayed or mice could be cured by the treatment with either the natural BRM or the synthetic BRM compositions and to see how the anti-tumor effects of different synthetic BRM compositions compare with those of natural BRM. Tumor sizes were measured every other day from day 4 after the tumor cell inoculation in two dimensions using a caliper, and the volume was expressed in mm³ using the formula: V=0.5 a×b², where a and b are the long and short diameters of the tumor, respectively. The treatments were terminated at day 21. Mean tumor volumes calculated from each measurement were then plotted in a standard graph to compare the anti-tumor efficacy of drug treatments to that of control. One day after the last treatment, tumors were excised from the animals and tumor weights were measured. A standard bar graph was used to demonstrate the differences in tumor weights with each bar representing mean tumor weight calculated from 8 animals.

The purpose of this study was to demonstrate the efficacy of a synthetic BRM composition based on the components presented in Table 1. Two embodiments of a synthetic BRM composition used in this study showed biological responses that were similar to the natural BRM with respect to TNF-α release. The in vivo nude mouse xenograft model of MDA-MB-231 breast adenocarcinoma has been tested using these two synthetic BRM compositions and their antitumor efficacy has been compared with that of natural BRM.

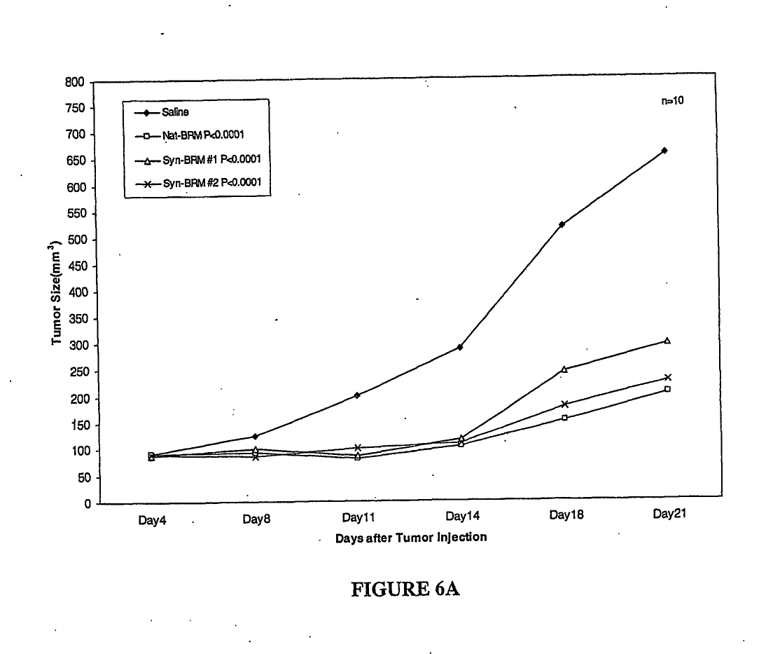

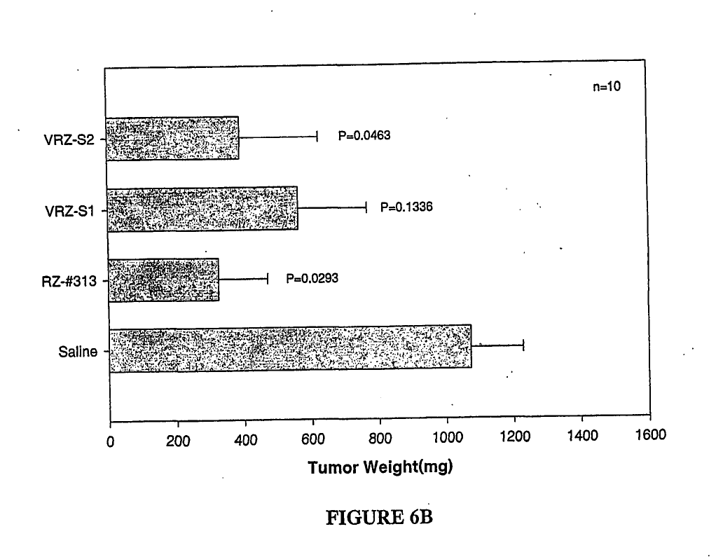

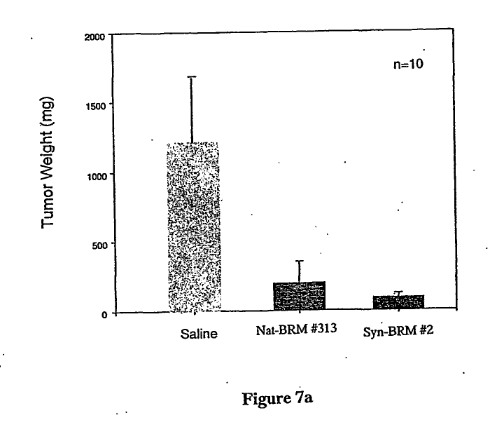

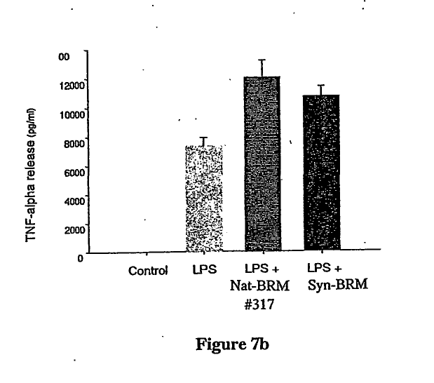

The results of the tumor growth curve and tumor weight measurements are shown in FIGS. 6(A) and (B). As illustrated, treatment with each of the two synthetic BRM compositions resulted in significant delay of tumor growth compared to saline control. The delay in tumor growth achieved with synthetic BRM #2 was as effective as that observed with natural BRM

### Example 8

### The Biological Activity of the BRM Composition

Studies are conducted to demonstrate the effect of the composition on cytokine release from peripheral blood mononuclear cells (PBMN) and/or U937 cells which is a stable line of pre-monocyte cells (American Type Culture Collection (ATCC), Rockville, Md.). These studies provide the basis for a standardized test for quantitatively evaluating the potency of a given batch of a synthetic BRM composition to evaluate the ability of the synthetic BRM composition to stimulate TNF-α production in the PBMN or U937 cells.

Whole blood is drawn from 5 healthy human subjects into heparinized Vacutainer tubes (Beckton Dickinson, Canada). PBMNs were isolated by gradient centrifugation on Ficoll-Hypaque (Pharmacia). The PBMNs are washed twice with phosphate-buffered saline (PBS), counted and resuspended in RPMI 1640 culture medium (Gibco Labs) at a concentration of 10⁶ cells/0.5 ml. These cells are cultured in 24-well, flat-bottomed tissue culture plates (Falcon, Becton, Dickinson). A 0.5 ml aliquot of the PBMN suspension is added to each well, which contains 50 ng lipopolysaccharide (LPS) (from E. coli), 10 μl fetal calf serum and 10-3001 μl of the composition of the invention (see Example 1). If necessary, the hyperosmolar effect of the composition is neutralized by adding distilled water to the culture wells at a volume equivalent to 10% of the volume of composition used. The total volume is then made up to 1 ml/well with RPMI. PBS is used as a control. The cells are cultured for 2, 6, 24, 48 and 72 hrs at 37° C. in a humidified 5% CO₂ incubator. At the end of each incubation period, the cells are harvested and cell-free culture fluids are obtained by centrifugation at 9000 rpm for 10 mins. The samples are then stored for up to 2 weeks at −70° C. until immunoassays, such as ELISA, are conducted to quantify the cytokines present.

Cytokine synthesis in the supernatants are measured after stimulating human PBMN with the BRM composition at volumes of 100 and 200 μL per well.

Cytokine synthesis in the supernatants is measured at 24 hrs at 37° C. after stimulating PBMNs with the BRM composition and LPS (or LPS alone as positive control), using volumes of 100 μl of the BRM composition per well. TNF is measured by a TNF-α ELISA kit (Endogen, Inc.), which detects a minimum level of 5 pg/ml of the cytokine. The other ELISA immunoassay kits that are used includ: IL-1α (Endogen, Inc.); GM-CSF (Endogen, Inc.); RFN-α (Endogen, Inc.); IL-2 (Advanced Magnetics, Inc.); IL-6 (Advanced Magnetics, Inc.); IL-1 (Advanced Magnetics, Inc.); IL-4 (R&D Systems); and IL-8 (R&D Systems). The results indicate that TNF is the major cytokine present in the supernatants, along with smaller amounts of IL-1β and GM-CSF. For example, a 40 μl dose of the BRM composition of stimulates the production and release of TNFα GM-CSF, and IL-1β.

Different deletion batches of the BRM composition are examined for their effect on LPS-induced release of TNF.

The PBMN-TNF assay as described above is standardized using 100 μl of the BRM composition and 50 ng of LPS. PBMNs from 3 different human subjects are obtained as described above and used the same day. The results of each of the three assays (using individual subject cells) are averaged to compensate for variations in response between different subjects. The analysis involves determining the amount of TNF-α released in RPMI media alone and in the presence of 50 ng LPS. The TNF-α released in the presence of 100 μl of the BRM composition in combination with 50 ng LPS is also determined. The TNF-α released in media is subtracted from the LPS value to obtain the TNF-α released in the presence of LPS alone. The media and LPS values are subtracted from the combined composition and LPS value to obtain the TNF-α released in the presence of the composition alone (reported in pg/ml). Accordingly, the TNF release assay serves to quantify the potency of the BRM composition.

The BRM composition can stimulate release of TNF-α from U937 cells, which are originally derived from a patient with histocytic lymphoma and display many characteristics of monocytes. U937 cells can be obtained from the ATCC. They are routinely maintained in RPMI-1640 medium (GIBCO, Grand Island, N.Y.) supplemented with 10% heat-inactivated fetal calf serum (FCS, GIBCO), 2 mM L-glutamine (ICN Biomedical Inc, Costa Mesa, Calif.), and 10 μg/ml Gentamycin Sulfate (SIGMA, Mississauga, Ontario, Canada) at 37° C., 5% CO₂. Passage of the U937 cells is performed every 34 days and seeding is at an initial concentration of 5×10⁵ cells/ml. The U937 cells can be stimulated to differentiate to monocytes by exposure to phorbol 12-myristate 13-acetate (PMA; Sigma Chemical Co., St. Louis, Mo.). The resulting monocytes have the capacity to release TNF upon stimulation, such as with the composition of Example 1, alone or in combination with LPS.

PMA is first dissolved in dimethyl sulfoxide (DMSO, SIGMA) at a concentration of 10 mM and then diluted 1000-fold with PBS to a stock solution concentration of 10 μM and stored at −20° C. U937 cell suspensions are centrifuged at 350×g for 10 mins at room temperature and reconstituted in fresh complete RPMI-1640 medium at a concentration of 2×10⁶ cells/ml. Cell viability is determined by trypan blue exclusion and is routinely greater than 95%. PMA is further diluted 500-fold with complete culture media to a concentration of 20 nM.

Aliquots of 0.5 ml of U937 cells (10⁶ cell/ml) are cultured in the presence or absence of 0.5 ml of PMA (20 nM) in 24-well, flat-bottom tissue culture plates (Becton Dickinson, Lincoln Park, N.J.) and incubated for 72 hrs at 37° C., 5% CO₂. The final concentrations per well are 5×10⁵ cells and 10 nM PMA.

After 72 hrs of incubation, 120 μl of media are removed and replaced by 100 μl of the composition of Example 1 and 10 μl of sterile deionized distilled water, in the presence or absence of 10 μl of LPS (5 ng/μl). After 24 hrs of incubation, any cells and particulate matter are pelleted by centrifugation at 350×g for 10 min and the resulting supernatants are stored at −20□C until they are assayed for TNF-α. BRM samples are tested on two separate occasions.

Two-site sandwich ELISAs are performed to quantify TNF-α in the U937 cell culture supernatants using TNF-α ELISA kits purchased from Endogen, Inc. (Cedarlane Laboratories, Hornby, Ontario). The protocol recommended by the manufacturer is used. Briefly, 100 μl of TNF-α standards and test samples are added to antihuman TNF-α pre-coated 96-well plates and incubated at 37° C., 5% CO₂ for 3 hrs. After extensive washing with washing buffer, 100 μl of antihuman TNF-α conjugated to alkaline phosphatase is added to plates and incubated at 37° C., 5% CO₂ for 2 hrs. After incubation, the plates are washed as described above and 100 μl of premixed TMB substrate is added to each well and the enzymatic color reaction is allowed to develop at room temperature in the dark for 30 min. Then 100 μl of stop solution is added to each well to stop the reaction and the plates are read using an SLT Lab Instrument ELISA reader at 450 nm. The detection limit of the assay is 5 pg/ml.

TNF values for U937 cells are determined as described for PBMN cells.

### Example 9

### Effects of the BRM Composition on T and B Lymphocytes in Culture

The growth of human lymphocytes is examined under carefully controlled conditions in the presence and absence of the BRM composition. Standard concentrations of lymphocytes are incubated in wells containing various concentrations of the composition. When normal T and B human lymphocytes are incubated with the composition in concentrations similar to those that are used clinically, there are no adverse effects as judged by trypan blue dye exclusion. Accordingly, the composition of the invention are non-toxic to normal T and B lymphocytes in culture.

The effect of the composition on the survival of human PBMN is also examined. PBMNs are incubated for 24 and 48 hrs in plastic microwell plates with various volumes of the composition and tissue culture medium. At the end of this period, the number of surviving cells is estimated by trypan blue dye exclusion.

The number of surviving cells fell at 24 and again at 48 hours in the presence or absence of the composition is indicative of the cytotoxicity to human PBMN.

The ability of the composition to stimulate lymphocytes can be evaluated in the following 3 indicator systems: 1) stimulation of lymphocyte DNA synthesis; 2) induction of lymphocyte-mediated cytotoxic function; and 3) induction of monocyte/macrophage-mediated cytotoxic function. These tests were chosen for the screen because they measure immunological functions that have been shown to be associated with different clinical parameters in patients with malignant disease. These indicators of immune function also can be modulated in cancer patients treated with different biological response modifying agents, such as IFN or IL-2.

### Example 10

### Pharmacodynamic Studies in Mice with the BRM Composition

Peritoneal macrophages are harvested from C57BL/6 mice 72 hours after intraperitoneal injection of 1.5 ml of 4% protease peptone. The macrophages are then stimulated in vitro with medium alone, 50 ng LPS, or BRM. Measurements of the stimulation are performed with respect to TNF (by ELISA) and NO (by spectrophotometric assay using the Greiss reagent) levels in duplicate experiments.

In vitro synergy of BRM with LPS for TNF-α release can similarly be addressed. Peritoneal macrophages were harvested from C57BL/6 mice after the same aforementioned treatment. The macrophages were then stimulated with 50 ng LPS alone or LPS with different dilutions of Nat-BRM. As above, TNF was determined via ELISA LPS alone induces about TNF-α release from mouse peritoneal macrophages in vitro.

In vitro synergy of BRM with LPS for nitric oxide (NO) is addressed in the same procedure as above, except NO is determined in the supernatant of the treated macrophages. As above, the assay for NO is spectrophotometric and uses a Greiss reagent. LPS causes some release of NO. BRM in synergy with LPS induces a marked increase in NO production. Nat-BRM by itself does not induce release of NO by macrophages.

In vitro synergy of BRM with IFN-γ for TNF-α release is studied using the same peritoneal mouse macrophages derived from C57BL/6 mice treated as above. Peritoneal mouse macrophages exhibit a baseline release of TNF-α after 24 hours of in vitro culture. The same macrophages stimulated with either LPS or IFN-γ release almost 3000 pg/ml of TNF-α.

In vitro synergy of BRM with IFN-γ for NO release is studied, using the same peritoneal mouse macrophages derived from C57BL/6 mice treated as above. LPS and IFN-γ alone each enhanced NO production.

In vivo production of TNF-α over 72 hours is studied on macrophages harvested from C57BL/6 mice that, prior to harvest, were treated with nothing, injected intraperitoneally 72 hours previously with 1.5 ml of 4% protease peptone, or injected intraperitoneally 72, 48, or 24 hours previously with 1.0 ml Nat-BRM diluted 1:10 in PBS. The macrophage monolayers are treated in vitro for 24 hours with IFN-γ, (50 μl/ml), LPS only (5 ng/ml), or the combination thereof. TNF and NO are determined as recited above.

The release of TNF-α from macrophages is examined in the absence of a stimulus or with IFN-γ, LPS, or LPS/IFN-γ after 24 hrs in vitro culture. When harvested macrophages are exposed to IFN-γ at 24 and 48 hrs prior to testing, they show a small increase in production of TNF-α. By contrast, harvested macrophages stimulated with LPS at 24 and 48 hrs, but not 72 hrs prior to testing, show enhanced release of TNF-α. Likewise, there is a synergistic effect of LPS and IFN-γ on harvested macrophages that were stimulated 24 and 48 hrs but not 72 hrs before testing.

### Example 11

### The BRM Composition has TNF-α Releasing Activity

This Example demonstrates, in summary, the following: (1) the composition has TNF-α releasing activity and the TNF-α releasing activity is not related to any contamination with endotoxin; (2) priming of macrophages enhances the ability of the composition to stimulate release of TNF-α; and (3) the hyperosmolarity of the composition is not responsible for TNF-α releasing activity.

To test whether an endotoxin effect is associated with the biological activity noted above for the composition of Example 1, further composition experiments are performed with polymyxin added to the reactants. Polymyxin inhibits the action of endotoxin on leukocytes

The osmolarity of different batches is determined using standard methods. The effect of the hyperosmolarity of the composition on TNF-α releasing activity is also studied

### Example 12

### Activation of Monocytes and Macrophages with the BRM Composition

BRM compositions will activate normal monocytes to demonstrate cytotoxicity towards the Chang hepatoma cell line, which is used to measure monocyte toxicity, and that the monocytes and macrophages from cancer patients (e.g., those afflicted with cancers of the cervix, ovaries, ear/nose/throat, and endometrium/uterus, and chronic myelogenous leukemia) which have been stimulated by the composition to attack and destroy tumor cells derived from the same patient.

The monocyte tumoricidal function is tested in the presence of the composition of the invention and the basic procedure for these experiments is outlined below. This procedure has been named the “Monocyte/Macrophage Cytotoxicity Assay to Cell Lines and Autologous Tumor Cells,” or “Cytotoxicity Assay” for short.

The method requires isolation of monocytes/macrophages, which is accomplished as follows: Venous blood is collected aseptically in heparinized Vacutainer tubes. Sterile preservative-free heparin is added to a final concentration of 20 units/ml. The blood is diluted 3:1 in Hanks balanced salt solution (HBSS), layered onto lymphocyte separation medium and centrifuged to obtain a band of peripheral blood mononuclear cells (PBMNs). After centrifugation, the mononuclear cell layer is recovered from the interface, washed twice in medium (medium is Roswell Park Memorial Institute [RPMI] 1640 media supplemented with 10% heat-inactivated fetal bovine serum, 50 units/ml penicillin, and 50 μg/ml streptomycin) and monocytes are enumerated by latex ingestion. Monocytes are isolated by adherence in 96-well plastic plates (for 2 hours at 37° C., followed by two cycles of washing with medium). Adherent cells are estimated to be greater than 90% monocytes. Wells containing adherent cells are incubated overnight in the presence of Nat-BRM (1:10-1:200 final dilution). Then, adherent cells are washed to remove Nat-BRM and incubated overnight with tumor cells. The tumor cells are maintained in medium in which endotoxin concentration is guaranteed by the manufacturer to be low and is non-stimulatory in the assay.

For studies using a standard cell line, ⁵¹Cr (chromium) labelled Chang hepatoma cells are used because this cell line is insensitive to natural killer cell cytotoxicity. These hepatoma target tumor cells are added to adherent cell monolayers at effector:target (E:T) cell ratios of 20:1 to 15:1. This E:T ratio is used because it falls well into the plateau range on a curve prepared by varying the E:T ratio from 5:1 to 30:1. After 24 hours, supernatants are collected and ⁵¹Cr release is quantitated as a representation of cytotoxicity (i.e. cell lysis). The percent specific cytotoxicity is calculated as:

$$\% specific release = \frac{E - S}{T - S} \times 100$$

In the equation above, E=CPM released from target cells in the presence of effector cells; S=CPM released from target cells in the absence of effector cells; T=CPM released from target cells after treatment with 2% sodium dodecyl sulfate.

For studies using autologous tumor cells, these cells are obtained from surgical biopsies, labelled with ⁵¹C, and used in the same way as the hepatoma cells described above.

Preparation of peritoneal and alveolar macrophages is done by the methods described in Braun et al., Cancer Research, 53, 3362-3365 (1993).

Using this protocol, the composition is found to cause monocytes from healthy donors to exert cytotoxicity toward the Chang hepatoma cell line. Subsequently, whether monocytes and macrophages from a cancer patient could be stimulated by the composition to attack and destroy their own particular tumor is investigated. Using similar protocols as described for the standard cell line (Chang hepatoma cells), monocytes and/or peritoneal macrophages from cancer patients are isolated. Peritoneal macrophages are isolated from peritoneal fluids collected at the time of laparoscopy.

### Example 13

### Monocyte/Macrophage Studies with SYN-BRM Composition

A number of comparative studies aimed at determining the dose response characteristics of the composition in stimulating monocyte/macrophage tumoricidal function are performed as well as testing different batches of the composition. The main emphasis of the studies is to demonstrate the capacity of the composition to simulate tumoricidal function in monocytes and macrophages from different anatomical sites of cancer patients. For these investigations, the following can be relied upon: (1) peripheral blood monocytes from cancer patients and control subjects; (2) alveolar macrophages from lung cancer patients and control patients with non-malignant lung diseases; and (3) peritoneal macrophages from patients with gynecological malignancies.

Dose response studies with different batches of the composition, all prepared in accordance with Example 1, are completed. These studies rely on peripheral blood monocytes to test the stimulatory activities of different doses and different batches of the composition. Each batch of the composition is tested without dilution (neat), a 1:10 dilution and a 1:50 dilution of material.

Tumoricidal function in peripheral blood monocytes is also evaluated. Tests are performed on 4 peripheral blood monocyte samples from control subjects. These tests utilize an optimal stimulating concentration of the composition (eg. 1:10 dilution) and an optimal stimulating concentration of IFN-γ plus LPS. The target cells in these studies are a cultured, NK-insensitive cell line, namely the Chang Hepatoma.

A test is also performed on 1 monocyte sample from a patient with cervical cancer. This test is important because the patient's own tumor cells are available to be used as target cells in the assay. As before, this test utilized an optimal stimulating concentration of the composition (eg., 1:10 dilution) and an optimal stimulating concentration of IFN-γ plus LPS. Also, the effector/target cell ratio is reduced to 15/1 to conserve patient tumor cells.

In the peripheral blood monocytes from control subjects, the composition stimulates monocyte tumoricidal function against the Chang Hepatoma cells at a level equal to or greater than the level elicited by an optimal stimulating concentration of IFN-γY+LPS. In the peripheral blood monocytes from a patient with cervical cancer, the composition stimulates tumoricidal function against the patient's own tumor cells at a level which exceeds that elicited by IFN-γ plus LPS.

Tumoricidal function in peritoneal macrophages from patients with gynecological malignancies can be tested. These tests are performed on peritoneal macrophage samples isolated from lavage fluids of patients with cervical cancer and patients with ovarian cancer. These tests are performed with the patient's own tumor cells as target cells in the assay. As before, an optimal stimulating concentration of the composition and an optimal stimulating concentration of IFN-γ plus LPS are compared. Also, the effector/target cell ratio can be reduced to 15/1 to conserve patient tumor cells.

These test results highlight the fact that the local tumor environment may be a determinant of the response of immune cells to immunological activators. In this case of cervical cancer, there is reduced pathological evidence of malignant disease within the peritoneal cavity and the development of tumoricidal function against the autologous tumor is better with IFN-γ and LPS combined than with the composition. The response against the patient's own tumor to IFN-γ and LPS combined is minimal at best, whereas the response to the composition was greater.

Tumoricidal function in alveolar macrophages from lung cancer patients and control subjects is tested. These tests can be performed on alveolar macrophage samples isolated from bronchoalveolar lavage fluids of a patient with non-small cell lung cancer and patients with non-malignant diseases of the lung. These tests utilize an optimal stimulating concentration of the composition and an optimal stimulating concentration of IFN-γ and LPS combined. The target cells in these studies were the Chang Hepatoma cells and the effector/target cell ratio is 20/1.

The results are consistent with the observation that alveolar macrophages from lung cancer patients are impaired in their development of tumoricidal function in response to conventional macrophage activators such as IFN-γ+LPS. The results show that the tumoricidal function of alveolar macrophages from lung cancer patients is greatly reduced compared to control subjects.

Accordingly, the composition can activate tumoricidal activity in alveolar macrophages.

The preliminary in vitro tests with the composition demonstrate that it is a macrophage activator. The material provided is able to elicit tumoricidal activity in a standard cytotoxicity assay against both an NK insensitive cell line and against freshly dissociated human tumor cells. The activity elicited is also found to be concentration-dependent in these tests. The capacity of the composition to active macrophage tumoricidal function in vitro is comparable to that of the best macrophage activating combination presently available, namely, IFN-γ and endotoxin (i.e., LPS) combined. As stated above, the capacity of the composition to elicit this level of tumoricidal function in the absence of endotoxin would be considered important biologically if the material is free of endotoxin contamination. The composition is free of endotoxin contamination when tested for pyrogens by the United States Pharmacoepeia (USP) rabbit pyrogen test.

As has been found for other macrophage activators, the activity of the composition in stimulating macrophage tumoricidal function varies with the source of the macrophages. It appears that the composition is an excellent activator of peripheral blood monocytes being equivalent to IFN-γ+LPS with normal donors and possibly superior to IFN-γ+LPS with cancer patient donors. Malignant disease has a significant impact on the development of monocyte tumoricidal function depending on the activator used (Braun et al., (1991)). One determinant of the biological activity of different macrophage activators in cancer patients monocytes is the sensitivity of the activator to arachidonic acid metabolism and the secretion by the cell of prostaglandins. From these initial studies with the composition, it appears that activity elicited with the compound is not sensitive to the inhibitory effects of prostaglandins. If prostaglandin insensitivity can be proven definitively for cancer patient monocytes stimulated with the composition, this would be considered important therapeutically because the effectiveness of many other biological activators is limited by prostaglandins. Preliminary studies with 2 specimens indicate that the composition may have good activity in peritoneal macrophages, particularly when malignant disease is present in the peritoneal cavity.

### Example 14

### Evaluation of Natural Killer (NK) Cell Infiltration in Mice Harboring Human Melanoma Xenografts

The mouse xenograft model of human melanoma was used in these studies to demonstrate the effect of treatment with a syn-BRM composition (Syn-BRM#4) on NK cell infiltration into tumors isolated from mice harboring tumors. The cell line used in this experiment to inoculate mice were human melanoma (C8161) cells, although any carcinoma cell line capable of tumor formation upon inoculation could be used. The tumor cell lines were grown as outlined above and SCID mice were inoculated with these cell lines as described in the previous examples.

A human melanoma cell line (C8161) was grown as monolayer culture in Minimum essential medium (−MEM) supplemented with 10% fetal bovine serum (FBS), 0.1 mM non-essential amino acid, 1.0 mM sodium pyruvate, 100 U/ml penicillin, 100 μg/ml streptomycin and 0.25 μg/ml amphotericin B and 2 mM L-alanyl-1-glutamine at 37° C. in an atmosphere of 5% CO₂ in air. The tumor cells were routinely subcultured twice weekly by trypsin-EDTA treatment. The cells were harvested from subconfluent logarithmically growing culture by treatment with trypsin-EDTA and counted for tumor inoculation.

Tumor Inoculation: An acclimation period of at least 7 days was allowed between receipt of the immunocompromised animal and its inoculation. Typically CD-1 or SCID mice were used. When the female mice were 6-9 (most typically 6-7) weeks of age, each mouse was subcutaneously injected in the right flank with 3-10 million human carcinoma cells in 0.1 ml of PBS. Inoculated animals were divided into equal sized treatment groups of 9-20 (typically about 10) mice each and treated daily with saline (0.2 ml/mouse/day, i.p.) or Nat-BRM (0.2 ml/mouse/day, i.p.).

The major endpoint was to determine the extent to which NK cells infiltrated tumors in the presence and absence of various syn-BRM compositions. Human tumor xenografts from mice treated with syn-BRM or saline were isolated after perfusion of mice with saline, homogenized in PBS and filtered with a Cell Strainer to produce a cell suspension. Dead cells and red blood cells were removed by gradient centrifugation with Histopaque-1077. Cells were washed twice with PBS and resuspended in culture medium supplemented with 2% FBS. Cells/sample (1×10⁶) were stained with anti-DX5, a NK cell specific antibody conjugated with FITC and/or anti-CD11b, a macrophage specific antibody conjugated with PE, and placed on ice for 30 minutes. The cells were subsequently washed once with medium and once with PBS, fixed with 1% paraformaldehyde in PBS and analysed by FACS (fluorescence activated cell sorting).

The results shown in FIG. 8 are from cells isolated from mice harboring C8161 human melanoma xenografts. This two-2 dimensional plot shows the cell numbers that stain with the macrophage specific antibodies in the y-axis and the NK specific antibodies in the x-axis. Animals treated with syn-BRM showed significantly increased NK infiltration into tumors compared to those treated with saline, suggesting a role for syn-BRM in NK cell activation and an association between the efficacy of syn-BRM and NK cell activity.

Accordingly, as shown in the aforestated in vitro studies, the composition of the present invention is demonstrated to be able to activate monocytes and macrophages to increase their immune system function.

### Example 15: Preparation of Nat-BRM

Bovine bile was collected from the gall bladders removed from healthy cows (both males and females) that were at least one and one-half years old. These cows were slaughtered for food use at a licensed and inspected abattoir. The slaughtered animals had been inspected and evaluated as healthy prior to slaughter and the gall bladders were separated from the livers and examined by a veterinarian to confirm that the gall bladders were free of parasites and evidence of infection, and thus suitable for use as a source of bile.

Gall bladders that passed this inspection were subjected to the following procedure: Gall bladders were wiped with a solution of 70% ethanol to sanitize the exterior of the bladders and bile was removed from the bladders with a syringe. The bile removed was visually examined in the syringe by the veterinarian to assure that it contained no blood or pus and was otherwise satisfactory. Bile from a healthy bovine is a greenish fluid substantially free of blood and pus. Fragments of livers, spleen, and lymph nodes were also collected from the animals whose bile was collected and the fragments were examined for the presence of parasites and other indications of disease.

For species that do not have a defined gall bladder (such as shark), bile is obtained directly from the hepatic organ.

Bile found to be satisfactory was transferred into a graduated amber bottle containing ethanol to give a 50% bile/50% ethanol solution by volume. The bile/ethanol solution was a greenish fluid substantially free of foreign material and tested positive for ethanol in accordance with methods recited at United States Pharmacopeia XXII, Part B (1994). These bottles were labeled with a lot number. Bile collected from a minimum of fifty animals was collected for each lot.

The bile/ethanol solution was then centrifuged at 4200 rpm for at least 2½ hours at 20±2° C. The supernatant liquid was decanted, filtered through a filter having, for example, a 2.5 μm retention, and checked for pH and ethanol content. The decanted liquid was then subjected to an activated charcoal treatment. The treated liquid was then monitored for Optical Density (OD) at 280 nm and conductivity. OD levels and/or conductivity levels outside specified ranges necessitated additional treatment of the liquid with activated carbon to achieve an OD and conductivity within specified ranges.

Following activated carbon treatment, the treated liquid filtered through a filter having, for example, a 2.5 μm retention, the ethanol was evaporated off (for example, by heating up to about 85° C.), and the treated liquid was concentrated to approximately one-eighth of the original bile/ethanol solution volume. The concentrated liquid was then cooled to 20-25° C., filtered through a filter having, for example, a 2.5 μm retention, and mixed with ethyl ether and the ether phase was discarded. This step can be repeated once. The aqueous phase was heated to remove residual ether (for example, by heating up to about 55° C. for about 10 hrs) and further reduced in volume to one-tenth of the original bile/ethanol volume by heating to around 80-85° C. The resultant composition was then tested for appearance, biological activity, and ethanol and ether content. The composition was a clear, yellowish solution, essentially free of foreign matter, and contained less than 10 ppm ethanol and less than 5 ppm ether.

Identity and purity were determined using reverse-phase high pressure liquid chromatography (reverse-phase HPLC). Potency is assayed using the monocyte/macrophage activation test referred to herein as the peripheral blood mononuclear cell-tumor necrosis factor assay (PBMN-TNF assay or, simply, TNF assay), as described in Example 2.

Initial batches of the composition were manufactured as a non-buffered liquid. Subsequent batches were manufactured as a buffered liquid, prepared by adjusting the pH of the composition to about 7.4±0.2, using hydrochloric acid (1%) solution and sodium hydroxide (1% solution), as well as using dibasic and monobasic sodium phosphate salts as buffers. Bioburden reduction was conducted in a steam autoclave at 104±2° C. for 60 mins. The bulk solution was filled into 5 ml or 10 ml sterile bottles and capped. The filled and capped bottles were subjected to three sterilization cycles by autoclaving them at 104° C.+2° C. for 60 mins followed by incubation at 35° C. for 23±1 hrs. Between each cycle of sterilization (autoclave plus incubation), samples were taken and tested for bioburden. Following the last cycle of sterilization, the bottles were visually inspected against a black and a white background to detect the presence of particulates.

Following inspection, the lot was sampled and tested for conformance to specifications. Tests included identity, sterility, pyrogenicity, endotoxin, bioassay, HPLC and general safety. The table below summarizes the data obtained for the various tests performed on the bile extract including normal ranges of data, where appropriate.

Characteristics of Batch Compositions Obtained in Accordance with Method of Example 15

| | BATCH # | BATCH # | BATCH # |
|---|---|---|---|
| FINAL PRODUCT TEST | BC0248 | BC0249 | BC0250 |
| Potency (pg/ml)* | 210 | 183 | 304 |
| Identity/Purity | Pass | Pass | Pass |
| Agrees with reference | | | |
| Safety (passes test | Pass | Pass | Pass |
| according to U.S. 81 | | | |
| CFR § 610.11) | | | |
| Pyrogenicity (temp. | Pass | Pass | Pass |
| increase shall | | | |
| not exceed 0.4° C.) | | | |
| Endotoxin 0.4 EU/ml | 0.25 | 0.25 | 0.25 |
| Sterility (no growth) | Pass | Pass | Pass |
| pH (7.40 ± 0.2) | 7.20 | 7.27 | 7.22 |
| Appearance - Visual | Pass | Pass | Pass |
| (clear, light yellowish | | | |
| liquid with little or no precipitate) | | | |
| Appearance - OD (passes test) | 1.34 | 1.38 | 1.85 |
| Osmolarity (<1000) | 877 | 854 | 832 |
| Solids (23 +/− 7 mg/ml) | 18 | 15 | 20 |
| Ethanol (not more than 10 ppm) | Pass | Pass | Pass |
| Ethyl Ether (not more than 5 ppm) | Pass | Pass | Pass |
| Conductivity (35 +/− 5 mMho) | 33 | 35 | 38 |

*Potency was measured with respect to monocyte/macrophage activation as described in Example 8; normal TNF-α release is at least 100 pg/ml.

Accordingly, Nat-BRM can be prepared from readily available sources of bile, using standard laboratory methods, resulting in a standardized final product.

### Example 16

### Analysis of Nat-BRM

Preparations of the Nat-BRM composition of Example 15 have been analyzed using methods known in the art to identify organic, inorganic and amino acid components of the composition.

### Example 16.1

### Analysis of Organic Components of Nat-BRM

LC/MS

Liquid chromatography coupled with mass spectrometry (LC/MS) was used to detect the presence of organic components in Nat-BRM, including 3-hydroxybutyric acid, lactic acid, acetic acid, creatine, creatinine, carnitine, taurine/taurocholic acid, choline, and urea (Table 3A). The presence of formic acid was also established using this methodology (Table 3B).

NMR Studies

1D and 2D NMR techniques were also used to identify of organic components in Nat-BRM. In deciding which compounds to analyse, biological relevance and profiles of already identified compounds and the theoretical structure and chemical shift range of the candidate compounds were considered. Biologically relevant compounds that fell within the molecular weight range found in Nat-BRM were considered. A large number of these compounds were metabolites found in the liver and biological fluids. Finally, compounds were screened based on their chemical shift profiles found in NMR databases (Aldrich, Sadtler, at the Chemistry library of University of Toronto and internet-based databases). A summary of the proton chemical shift assignments for identified compounds in Nat-BRM is given in Table 5.

Pre-treatment and preparation of NMR samples: Unless otherwise indicated, untreated Nat-BRM was used to acquire NMR data. For 2-dimensional NMR spectroscopy, Nat-BRM was completely dried by low-heat evaporation and re-dissolved in deuterated water. Pure authentic compounds were added into Nat-BRM, and resonance peaks compared to unknown peaks in order to determine if the spiked compound is present in Nat-BRM. To compare the chemical shifts of peaks from Nat-BRM with those of candidate compounds, authentic standard solutions (mixed or single) were prepared (10 mM in deuterated dd-water). The pH and osmolarity of the standard were adjusted to that of Nat-BRM prior to NMR measurements. To minimize data acquisition time and cost, some standards were mixed. Samples for NMR analysis were prepared by mixing 120 μl of each standard solution with 500 μl of Nat-BRM and 180 μl D₂O (containing 0.75% TSP, 3-(trimethylsilyl)-tetradeutero sodium propionate).

NMR data acquisition: 1-dimensional and 2-dimensional proton spectra (COSY (Correlation Spectroscopy), TOCSY Crotal Correlation Spectroscopy), and NOESY (Nuclear Overhauser Enhancement Spectroscopy) for correlation spectroscopy) were obtained using state-of-the-art high field-strength 400, 500, 600 MHz NMR spectrometers at the University of Guelph or the University of Toronto, Ontario, Canada NMR spectra of one-dimensional ¹H, ¹³C and ³¹P, and two-dimensional COSY, TOCSY, HSQC, and HMBC were recorded and analyzed.

Spectral analysis and interpretations: Spectral data processing and analyses were carried out using PC-based NMR computer software. Raw data, acquired from service laboratories, were subjected to transformation, phasing, baseline correction and chemical shift referencing. Spectra were aligned with each other, expanded for better viewing, and plotted. When required, peak integration was carried out to quantify peak areas. These analyses allowed evaluation of chemical shift comparison between resonances in Nat-BRM and those in samples of standards. Splitting patterns, ratios of integrals of peaks, lot-to-lot consistency and pH dependence on chemical shift of individual peaks were also used to aid in assignment.

Results of the NMR Analyses:

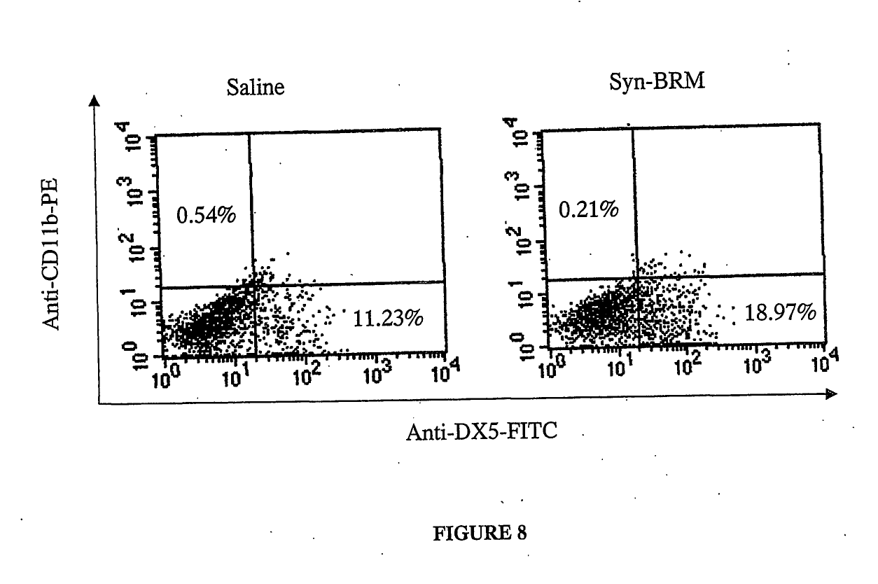

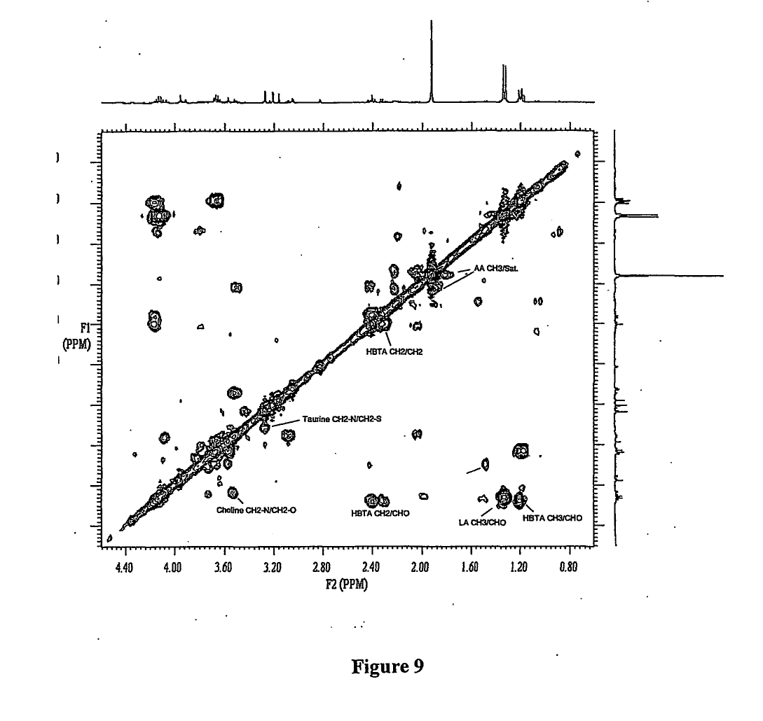

1 3-hydroxybutyric acid peaks were identified at 1.20 ppm for CH₃, 2.31 and 2.41 ppm for CH₂—CO, and 4.14 ppm for CH—O. The cross peaks of 3-hydroxybutyric acid in its proton group connections were obvious in 2D COSY (FIG. 9) and TOCSY spectra Therefore 3-hydroxybutyric acid was identified as a component of Nat-BRM.

2 Lactic acid peaks were identified at 1.33 ppm for CH₃, and 4.12 ppm for CH—O, which were confirmed by COSY experiment. Therefore lactic acid was identified as a component of Nat-BRM.

3 A peak at 1.93 ppm was assigned to a methyl group of acetic acid, which was surrounded by its two satellite peaks (at +/−0.1 ppm) identified in the COSY (FIG. 9) experiment. Therefore acetic acid has was identified as a component of Nat-BRM.

4 Peaks were assigned at 3.22 ppm to (CH₃)₃—N, one at 3.54 ppm to CH₂—N, one at 4.10 ppm to CH₂—O, identifying choline as a component of Nat-BRM.

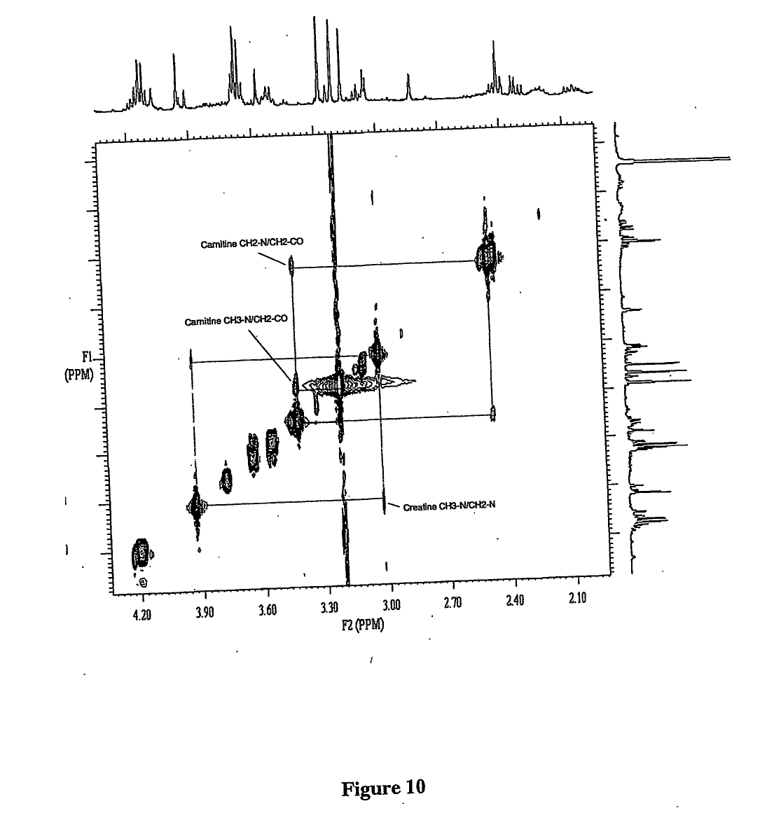

5 Creatine peaks were identified at 3.04 ppm for CH₃—N, and 3.94 ppm for CH₂—N. The spatial connection between these two groups was observed from their NOE cross peak in the NOESY, spectrum from NMR spectra (FIG. 10) thus identifying creatine as a component of Nat-BRM.

6 Creatinine peaks were assigned to a peak at 3.05 ppm for CH₃—N, and one at 4.07 ppm for CH₂—N. Thus creatinine has been identified as a component of Nat-BRM.

7 The CH₂—N peak of creatine was initially assigned to a strong peak at 3.97 ppm (3.96 ppm in the new spectra), however this did not superimpose with the peak in the spectra of the standard used to add creatine to Nat-BRM. Hence the relatively strong peak at 3.96 ppm was re-examined and identified as glycolate. The CH₂—N peak of creatine was re-assigned to an adjacent minor peak at 3.94 ppm.

8 Carnitine resonance peaks were relatively weak. The (CH₃)₃—N peak was detected at 3.23 ppm, and the CH₂—N peak was at 2.43 ppm which overlapped with CH₂—CO peak of 3-hydroxybutyric acid. Initially, the peaks from CH₂—CO and CH—O of carnitine could not be identified as they overlapped, overwhelmingly, with the peaks from taurine CH₂—S at 3.43 ppm and the residual solvent (water) peak at 4.74 ppm, respectively. Two NOE cross peaks between (CH₃)₃—N and CH₂—CO, and CH₂—N and CH₂—CO were found in the NOESY spectrum (FIG. 10) and the assignment of carnitine was confirmed.

9 Peaks from glycerol are expected at 3.56 and 3.65 ppm for CH₂—O, and 3.78 ppm for CH—O based on the spectra from the spiked authentic sample. Only a single CH₂—O peak at 3.65 ppm was observed in the spectra of Nat-BRM, in a region overlapped with ethanol CH₂ peaks. Since LC/MS studies demonstrated very low to undetectable levels of glycerol in Nat-BRM during method development studies, it is possible that Nat-BRM contains only trace amounts of glycerol.

10 Peaks at 3.28 and 3.44 ppm have been assigned to CH₂—N and CH₂—S of taurine, respectively. Peaks at 3.11 and 3.51 ppm were originally assigned to the CH₂—N and CH₂—S moeties of taurine. Using Nat-BRM with taurocholic acid added to it, it was ascertained that these peaks belonged to the taurine group in the taurocholic acid molecule.

11 A unique peak at 5.78 ppm was assigned to the NH₂— of urea. This was confirmed by an increase in the peak from a Nat-BRM sample after standard urea solution was added to it.

12 Previously identified peaks of phosphorylcholine and methylhydantoin did not exactly coincide with those of the phosphorylcholine and methylhydantoin standards. Additional experiments revealed that a 4.08 ppm phosphorylcholine peak (4.07 in the current study) was more properly assigned to creatinine. Other peaks previously assigned to phospholylcholine became unidentified peaks consistent with LC/MS results in which these compounds were ‘absent’ or at background levels in the Nat-BRM samples tested.

In summary, analysis of the data from NMR studies conformed the presence of 9 major components of Nat-BRM.

Further NMR Studies

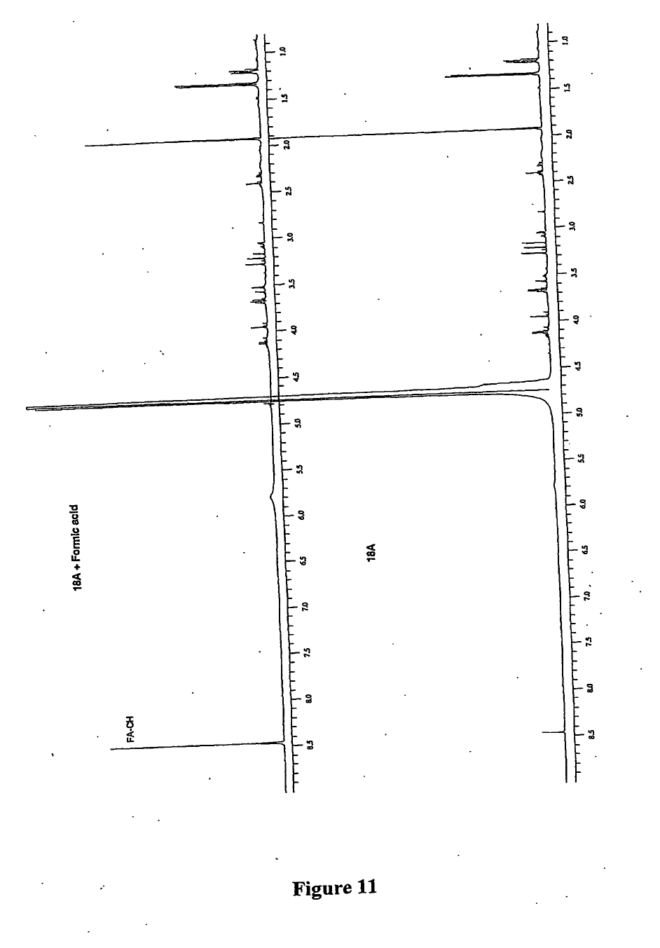

It was previously determined that most of the components in Nat-BRM are of low molecular weight (less than 1000 Da). In addition, all the identified organic components can be viewed as metabolites from a variety of pathways. To identify further unknown components of the Nat-BRM composition small molecular weight compounds can be considered that are part of the metabolic pathway of some of the already identified components, or are related to known components by structure or degradative pathway. In addition, published NMR studies on biological fluid samples and literature information on metabolic pathways (in particular liver metabolites), have been used to determine which candidate components to investigate (summarized in publications and books such as: E. Lynch, et al. J. of Inorganic Biochemistry, (1999), 73, 65-84; Lehninger's Principles of Biochemistry, Worth Publishers, Inc., New York, 1982). For example, formic acid is a compound whose NMR profiles, from NMR databases, coincided with unknown peaks in Nat-BRM. Furthermore, a literature search revealed this compound is found downstream of phosphoryl choline and choline in the biosynthetic pathway of glycine (Scheme 1 in Descampiaux et al, (1997) Chem. Res. Toxicol. 10: 3440). As a result, formic acid was added to Nat-BRM and 1D proton spectra were recorded in order to observe whether any resonance peaks matched an unidentified peaks. The addition of formic acid increased the height of an unidentified peak, and accordingly, formic acid was assigned to a peak at 8.46 ppm, (FIG. 11).

In addition, the testing for taurocholic acid (TC) matched peaks at 0.89, 0.93, 1.55, 2.04, and 2.21 ppm, which were typical resonance peaks for C18, C19, C20, C22, and C23 from cholic salts. Because those peaks were connected with two other typical taurocholic acid peaks (C25 and C26) at 3.09 and 3.52 ppm observed from the COSY (FIG. 9) and TOCSY spectra, the main cholic salt is likely taurocholic acid.

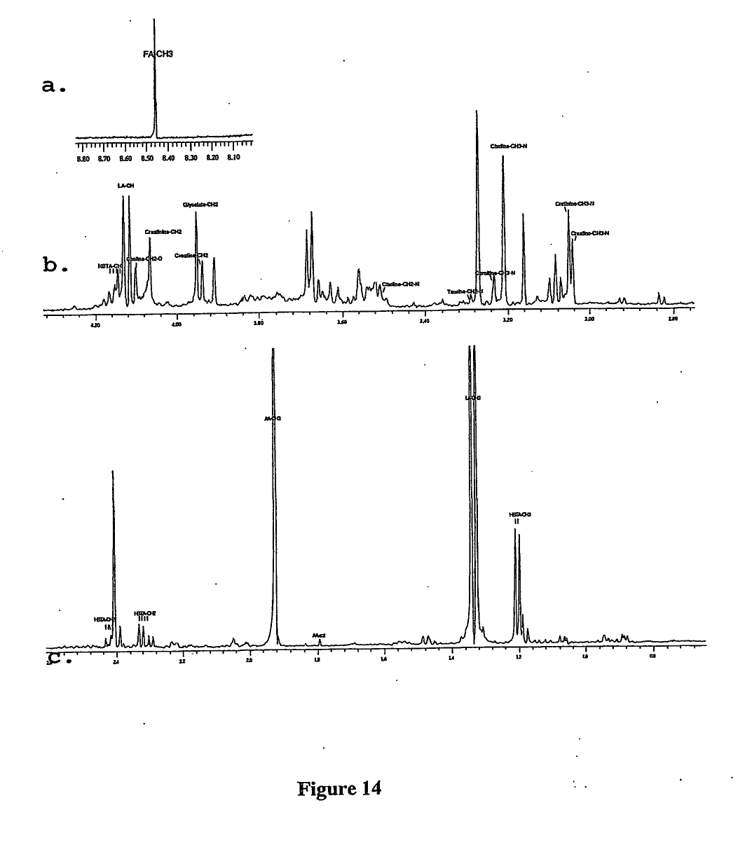

A typical proton NMR spectrum of Nat-BRM with the assignments is shown in FIG. 14. The NMR proton chemical shifts of identified compounds of Nat-BRM are summarized in Table 4, and the major peak assignments are summarized in Table 5.

### Example 16.2

### Analysis of Inorganic Components of Nat-BRM

Two lots each of Nat-BRM manufactured in two different facilities (the Dalton and Imutec facilities) were tested using standard methods known in the art for the presence of inorganic components. The detection limit for metals was 0.05 ppm, and for ions was 1.0 ppm. See Table 6 for the results of this analysis. Tungsten, barium, nickel, strontium, copper and manganese, if present, were below the detection limit.

### Example 17

### In Vivo Evaluation of Syn-BRM Efficacy in Mice Harbouring a Human Tumour Xenograft

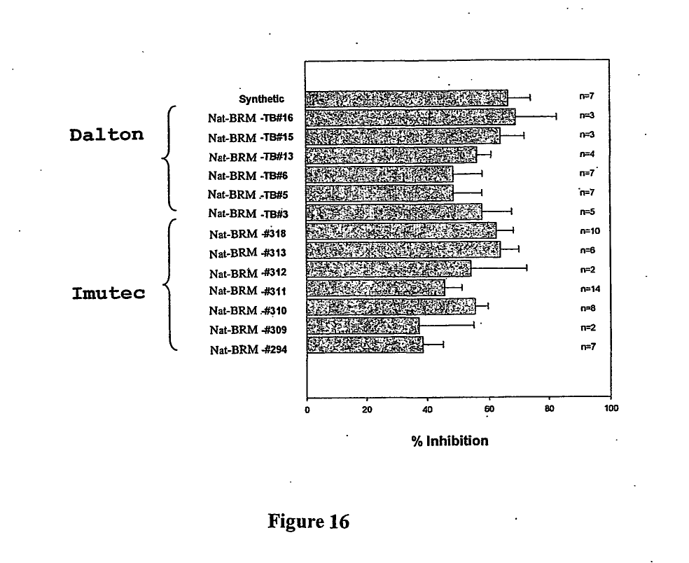

As described in Examples 2-7, and Example 18 the animal models such as the tumour xenograft model may be used to assess the antitumour activity of BRM compositions. FIG. 16 summarizes the results of several mouse xenograft experiments in which various lots of Nat-BRM and a Syn-BRM composition were shown to have comparable activity in vivo.

### Example 18

### Evaluation of Natural Killer (NK) Cell Infiltration in Nat-BRM-Treated Mice Harbouring Human Tumour Xenografts

In addition to a mechanism of direct contact (e.g. phagocytosis) for killing tumor cells, macrophages may exert their anti-tumor activity by a paracrine mechanism in which secretion of inflammatory cytokines, including TNF-alpha and IL-12, recruit and activate cytotoxic lymphocytes, such as NK cells, at the tumor site. To assess whether a paracrine mechanism of action is functional during Nat-BRM treatment, tumors were isolated from saline- and Nat-BRM-treated mice and the number of infiltrated NK cells was assessed first by immunohistochemistry and then by flow cytometry.

In collaboration with Dr. Hermon Yeger, of the Research Institute at the Hospital for Sick Children, Toronto, we have been using an immunohistochemistry approach to search for changes in several potential biomarkers, including markers for differentiation, proliferation, NK and macrophage cell infiltration and apoptosis in xenografted tumors. Tumor tissue sections from excised human tumor xenografts in mice were analyzed and no measurable difference between tumors from saline and Nat-BRM treated mice was observed for a number of markers. These observations may indicate that changes in numbers of infiltrating macrophage and NK cells may be relatively small and below the detection sensitivity of the method. As an alternative to immunohistochemistry, we are currently using flow cytometric techniques to identify biological marker(s) in response to Nat-BRM treatment. In undertaking this approach we reasoned that flow cytometric methods would be more sensitive in detecting changes in the number of infiltrated immune cells than are histological methods.

Analyses of infiltrated macrophage/monocyte (CD11b positive cells and NK) cells and assessment of cell cycle condition of tumor cells from Nat-BRM-treated tumors are ongoing. Preliminary results are described below.

2.2.2 FACS (Fluorescence Activated Cell Sorting) Methodology

Human tumor xenografts from mice treated with saline or Nat-BRM were isolated after perfusion of mice with saline. Isolated tissues were homogenized in PBS and filtered with a Cell Strainer to produce a cell suspension. Dead cells and red blood cells were removed by gradient centrifugation with Histopaque-1077. Cells were washed twice with PBS and resuspended in culture medium supplemented with 2% FBS. 1×10⁶ cells/sample were stained with anti-DX5 (NK specific) antibody conjugated with FITC and/or anti-CD11b (macrophage specific) antibody conjugated with PE, on ice for 30 minutes. The cells were subsequently washed once with medium and once with PBS, fixed with 1% paraformaldehyde in PBS and then analyzed by FACS. The results are presented as 2 dimensional plots of the cell numbers that stain with the macrophage specific antibodies in the y axis and the NK specific antibodies in the x axis.

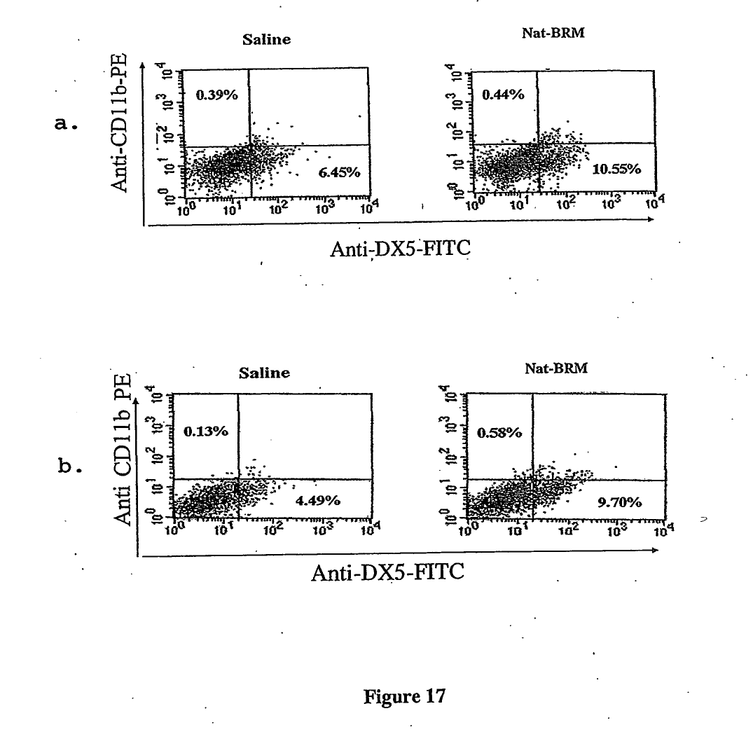

Preliminary experiments indicate that the population of infiltrated NK cells in human melanoma (C8161) xenograft tumors increased from 6.45%, in the saline-treated group, to 10.55% in the Nat-BRM treatment group (FIG. 17a). The C8161 experiments have been repeated and the averages of three experiments are in close agreement with the data in FIG. 17a. Hence, the average number of NK cells increased from 6.85% in saline-treated tumors to 10.92% in Nat-BRM-treated tumors, which is an increase of 59.4%. In a separate experiment, using human pancreatic (Capan-1) xenograft tumors, the population of infiltrated NK cells increased from 4.49%, in the saline-treated group, to 9.7% in the Nat-BRM treatment group (FIG. 17a). These results are consistent with the hypothesis that Nat-BRM immune modulation is mediated, at least partially, by NK cells, in vivo. However, the population of macrophages in the tumor tissues was too low to be analyzed accurately by flow cytometry. These data indicate that Nat-BRM-activated macrophages recruit and/or activate NK cells in tumor tissues.

In addition to the above approach, we are exploring the possibility of using an RT-PCR method to examine the expression of TNF-alpha, IL-12, IFN-gamma and iNOS in cells from Nat-BRM-treated tumors.

### Example 19

### In Vivo Evaluation of Macrophage Involvement in Nat-BRM Antitumour Effects

A study is currently under way that will differentiate between macrophage-mediated and NK-mediated immune responses after Nat-BRM treatment Human tumor xenografts in mice coupled with a macrophage depletion technique and NK-deficient mice (SCID/beige) will be used to assess the respective contributions of macrophage and NK cells in Nat-BRM-mediated tumor suppression. We have recently developed the macrophage depletion technique and preliminary results are presented below.

2.2.4 Macrophage Depletion Methodology

Selective depletion of macrophages in vivo was performed using the liposome/C12 MBP technique essentially as described by Van Rooijen and Sanders, 1994. Briefly, 200□l of liposomes prepared with C12 MBP were injected (i.v.) the day before transplantation of tumor cells. Additional injections, every three days, at the same dosage, followed tumor implantation. Mice bearing tumors were randomly separated into different groups for treatment with the indicated drugs. Treatment schedules were similar to the standard xenograft experiments with Nat-BRM treatment employed at our laboratory.

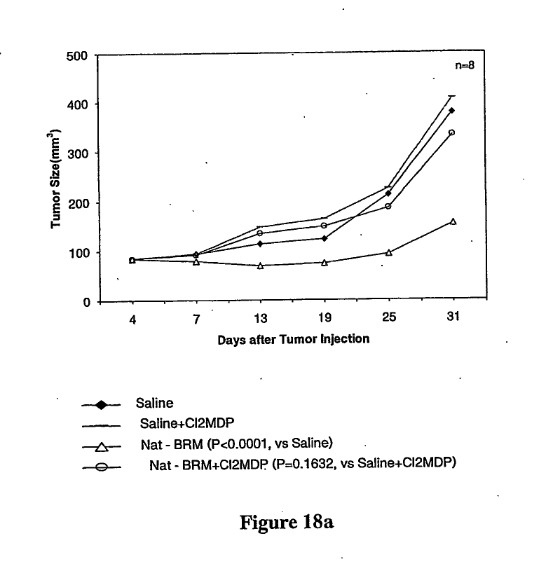

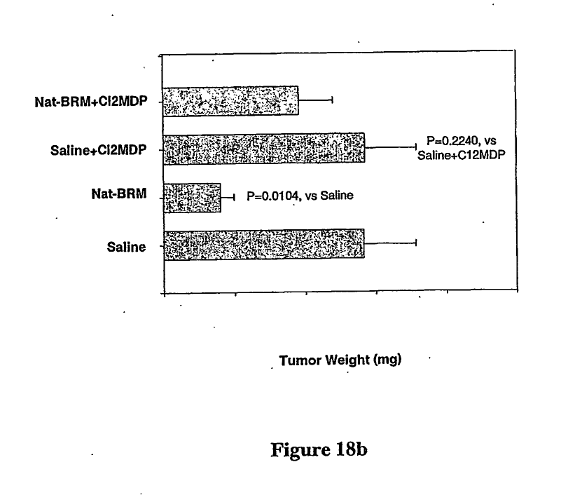

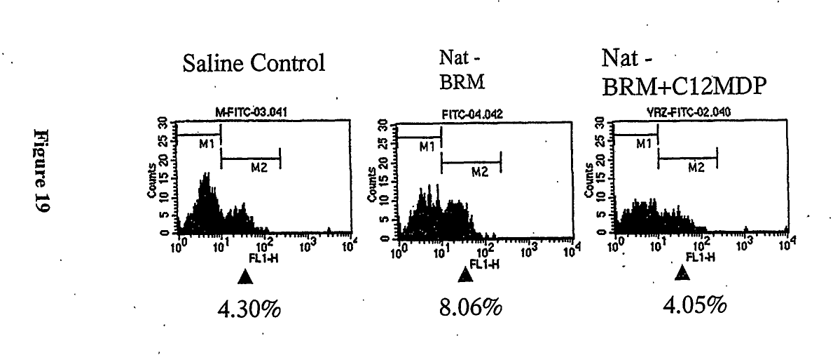

Results from an experiment examining the anticancer effect of Nat-BRM on the development of human melanoma tumors in mice treated with C12MDP, a chemical that selectively destroys macrophages, are presented in FIG. 18. The results indicate that the anti-tumor effect of Nat-BRM was significantly compromised after macrophage depletion, suggesting an important role for macrophages in Nat-BRM-mediated antitumor efficacy. To assess the relationship between macrophages and NK cell infiltration into tumor xenografts, mice were depleted of macrophages, injected with C8161 human melanoma cells and treated with saline or Nat-BRM and NK cell infiltration into tumors was assessed as in FIG. 17. Depletion of macrophages results in loss of Nat-BRM-induced increase in NK cell infiltrates into tumors suggesting that macrophages are involved in NK migration to tumors (FIG. 19).

In addition to investigating the role of macrophages and NK cells in the anti-tumor activity of Nat-BRM, we are investigating the role of IL-12 and/or TNF-α by neutralizing these factors using mono-specific antibodies followed by testing in xenograft models. We anticipate that the results of these experiments would be available by July, 2002. In addition to antibody neutralization we are examining the possibility of testing Nat-BRM efficacy in TNF-α or IL-12 knockout mice.

Moreover, we are attempting to develop cytotoxicity assays to examine the role of macrophage/monocyte interactions with a number of effector cells (NK cells or Th1 sub-type T lymphocytes). The effects of these interactions on Nat-BRM-mediated anti-cancer activity will be assessed by examining phagocytosis and release of inflammatory cytokine/mediators such as TNF-α, IFN-γ, IL-12 and perforin.

### Example 20

### In Vivo Evaluation of Efficacy of Nat-BRM in Mice Harbouring Human Tumour Xenografts

The mouse xenograft model of neoplasia was used in these studies to demonstrate the effect of treatment with a Nat-BRM composition on tumor growth in mice. For comparison, separate groups of mice were treated with saline (control), a conventional chemotherapeutic drug or concurrently with a combination of a Nat-BRM composition and a chemotherapeutic drug.

A human carcinoma cell line was grown as monolayer culture in Minimum essential medium (−MEM) supplemented with 10% fetal bovine serum (FBS), 0.1 mM non-essential amino acid, 1.0 mM sodium pyruvate, 100 U/ml penicillin, 100 μg/ml streptomycin and 0.25 μg/ml amphotericin B and 2 mM L-alanyl-1-glutamine at 37° C. in an atmosphere of 5% CO₂ in air. The tumor cells were routinely subcultured twice weekly by trypsin-EDTA treatment. The cells were harvested from subconfluent logarithmically growing culture by treatment with trypsin-EDTA and counted for tumor inoculation. The cell lines used in the experiments herein are listed hereafter, though any carcinoma cell line capable of tumor formation upon inoculation could be used:

pancreatic adenocarcinoma (BxPC-3) (a gemcitabine-resistant cell line)

melanoma (A2058)

melanoma (C8161)

breast adenocarcinoma (MDA-MB-231)

prostate carcinoma (PC-3)

ovary adenocarcinoma (SK-OV-3)

large cell lung adenocarcinoma (H460)

small cell lung carcinoma (H209).

Tumor Inoculation: An acclimation period of at least 7 days was allowed between receipt of the immunocompromised animal and its inoculation. Typically CD-1 or SCID mice were used. When the female mice were 6-9 (most typically 6-7) weeks of age, each mouse was subcutaneously injected in the right flank with 3-10 million human carcinoma cells in 0.1 ml of PBS. Inoculated animals were divided into equal sized treatment groups of 9-20 (typically about 10) mice each and treated daily with saline (0.2 ml/mouse/day, i.p.), Nat-BRM (0.2 ml/mouse/day, i.p.), a chemotherapeutic drug, or concurrently with Nat-BRM (0.2 ml/mouse/day, i.p.) and a chemotherapeutic drug. The drug doses used in the experiments herein are listed hereafter, though any chemotherapeutic drug(s) or other anticancer agent(s) could be used:

gemcitabine (100 mg/kg in 0.1 ml saline/mouse/3 day, i.v.)

dacarbazine (DTIC) (80 mg/kg in 0.1 ml saline/mouse/day, i.p.)

taxol (10 mg/kg/week, i.v.)

5-fluorouracil

taxotere

cisplatin

mitoxanthrone (i.v.)

Tumour sizes were measured every other day in two dimensions using a caliper, and the volume was expressed in mm³ using the formula: V=0.5 a×b², where a and b are the long and short diameters of the tumor, respectively. Mean tumor volumes calculated from each measurement were then plotted in a standard graph to compare the anti-tumor efficacy of drug treatments to that of control. A day after the last treatment, tumors were excised from the animals and their weights were measured. The data are displayed as a tumour growth curve, and a bar graph showing mean tumor weights.

TABLE
Mouse xenograft experiments with Nat-BRM compositions
and Nat-BRM combinations

| FIG. | Human | | Mouse | | combination | # mice with total |
|---|---|---|---|---|---|---|
| # | carcinoma | cell line | strain | drug | expt | tumor regression |
| 20, 21 | pancreatic | | CD-1 | gemcitabine | — | BRM: 4 (of 9) |
| 22, 23 | pancreatic | SU.86.86 | CD-1 | gemcitabine | gemcitabine | |
| 24, 25 | melanoma | A2058 | CD-1 | dacarbazine | dacarbazine | |
| 26, 27 | melanoma | C8161 | CD-1 | — | dacarbazine | comb: 5 (of 10) |
| 28, 29 | breast | MDA-MB- | CD-1 | Taxol | Taxol | |
| 30, 31 | breast | MDA-MB- | CD-1 | Taxol | Taxol | BRM: 2; comb: 5 (of 10) |
| 32, 33 | prostate | PC-3 | SCID | mitoxantrone | — | |
| 34 | pancreatic | BxPC-3 | CD-1 | 5-fluorouracil | 5-fluorouracil | comb: (5 of 10) |
| 35 | pancreatic | SU.86.86 | CD-1 | 5-fluorouracil | 5-fluorouracil | |
| 36, 37 | prostate | DU145 | SCID | mitoxantrone | — | |
| 38 | ovarian | SK-OV-3 | CD-1 | cisplatin | cisplatin | |
| 39 | ovarian | SK-OV-3 | CD-1 | taxol | taxol | |
| 40, 41 | lung, large | H460 | CD-1 | taxotere | taxotere | |
| | cell | | | | | |
| 42, 43 | lung, small | H209 | SCID | — | — | |
| | cell | | | | | |

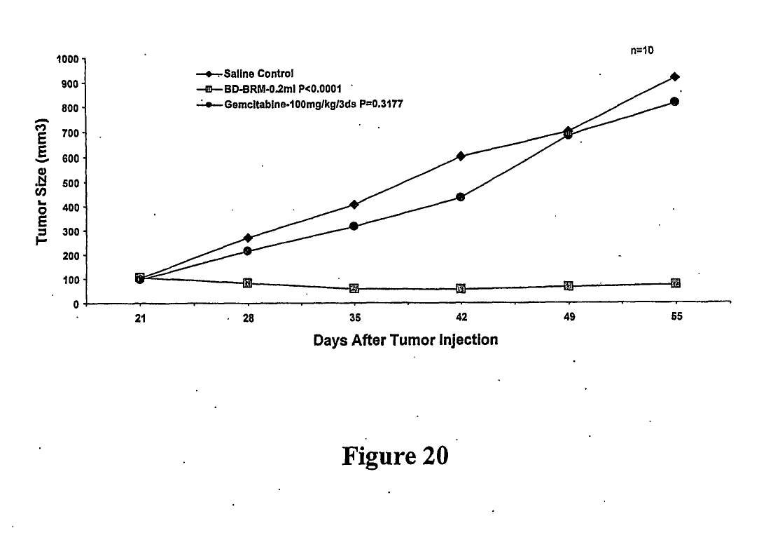

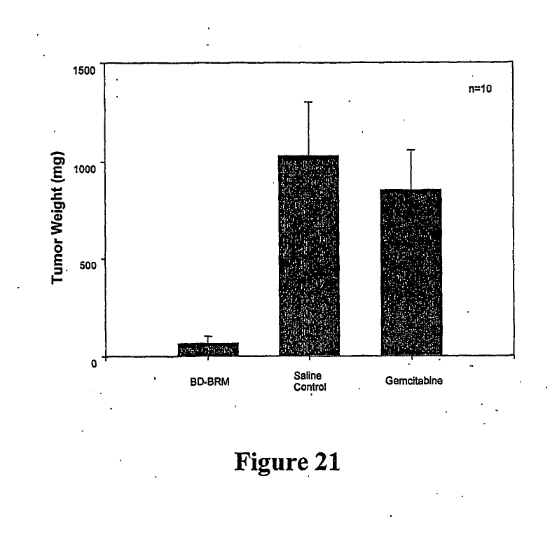

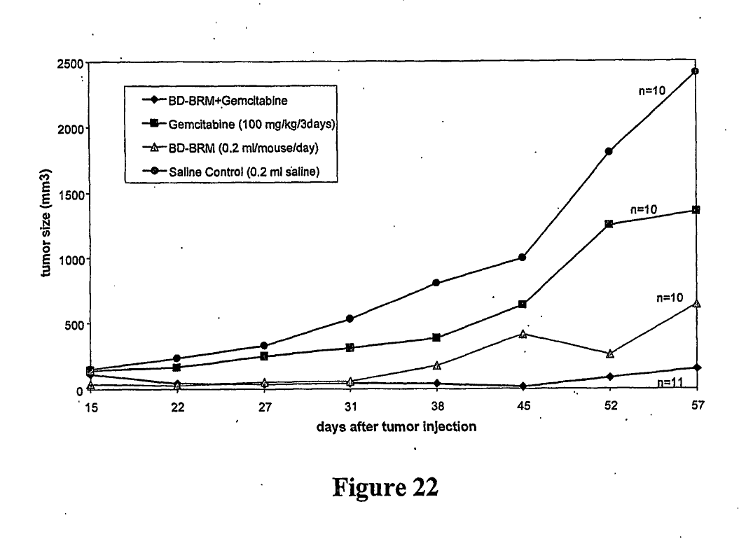

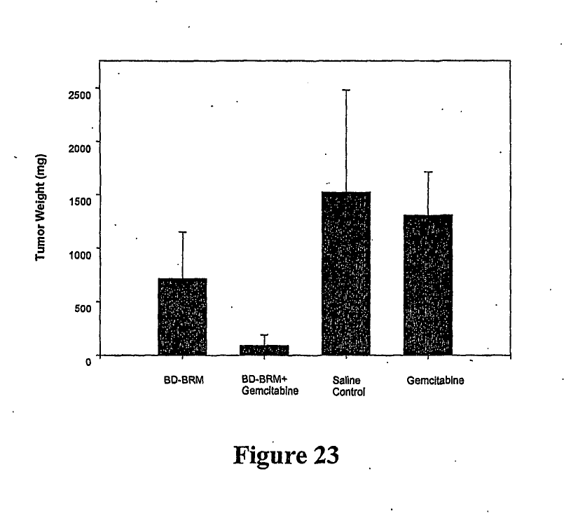

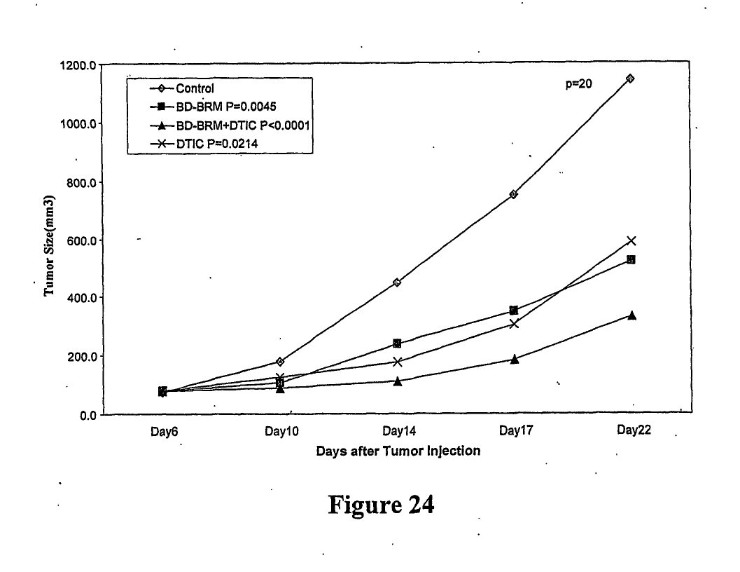

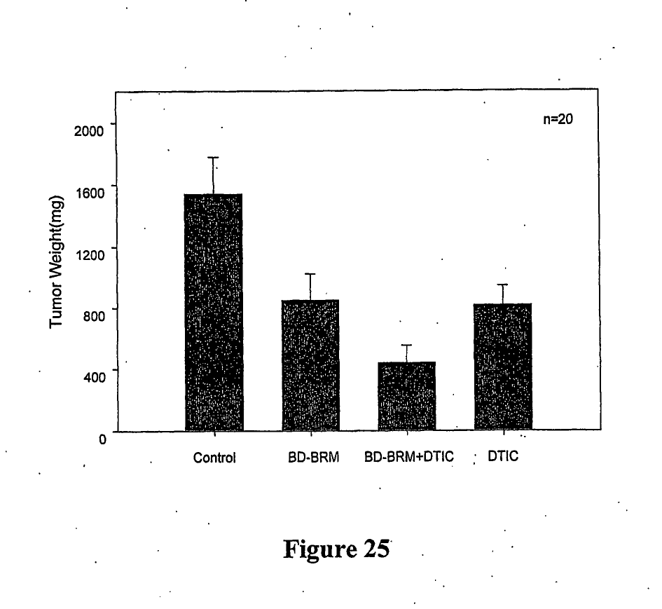

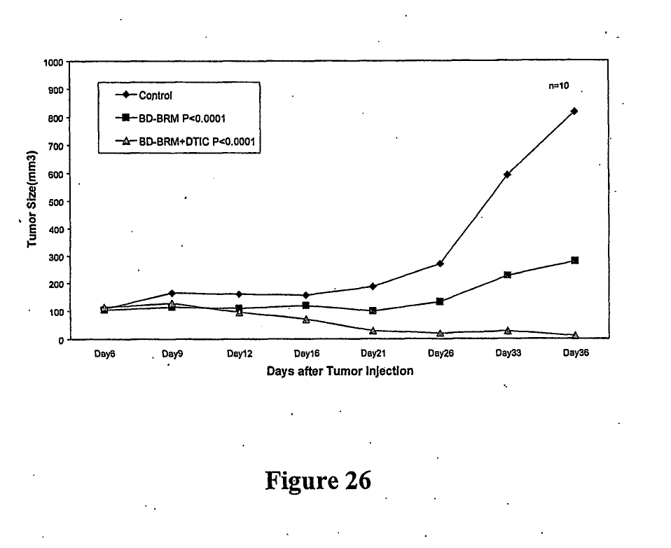

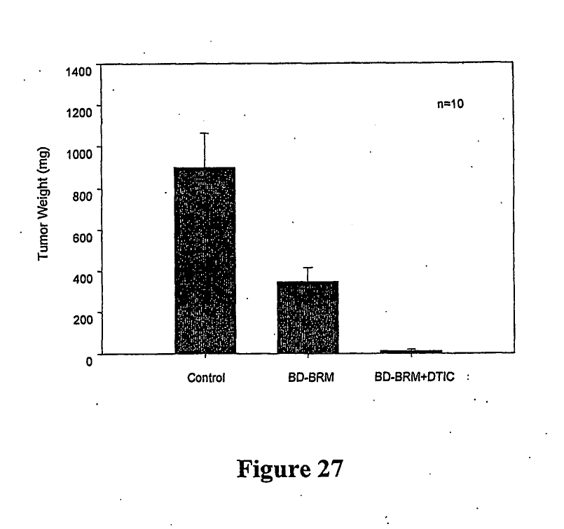

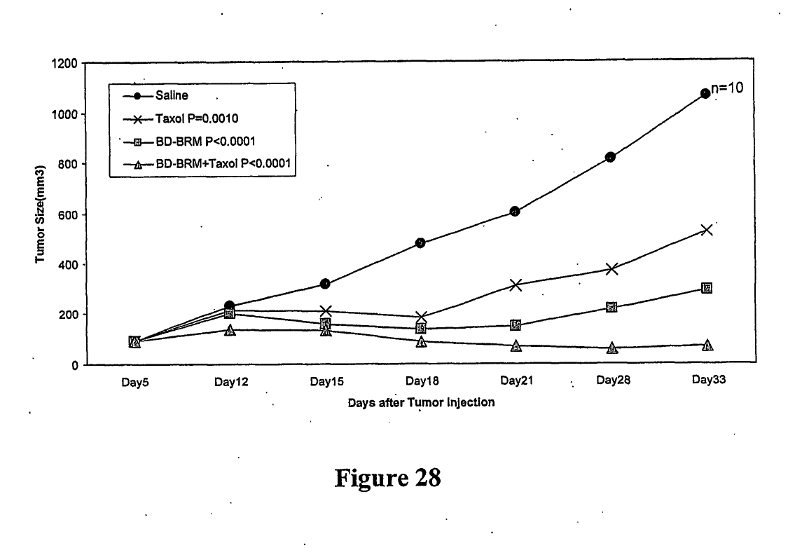

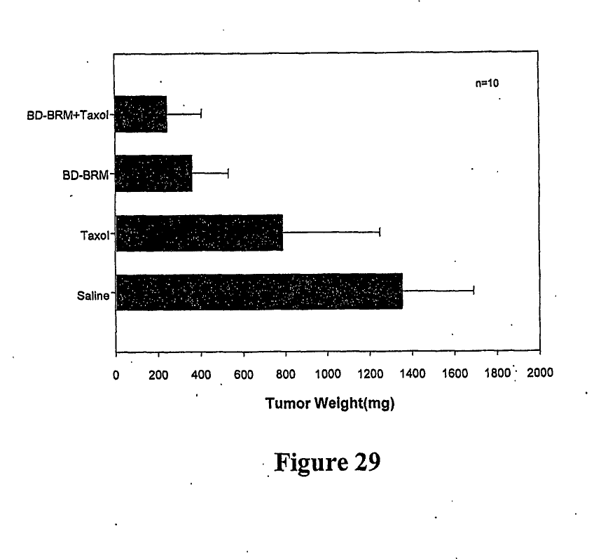

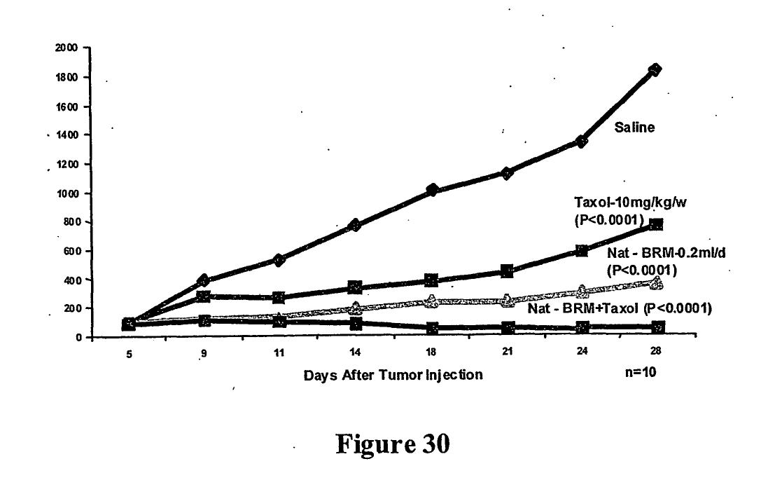

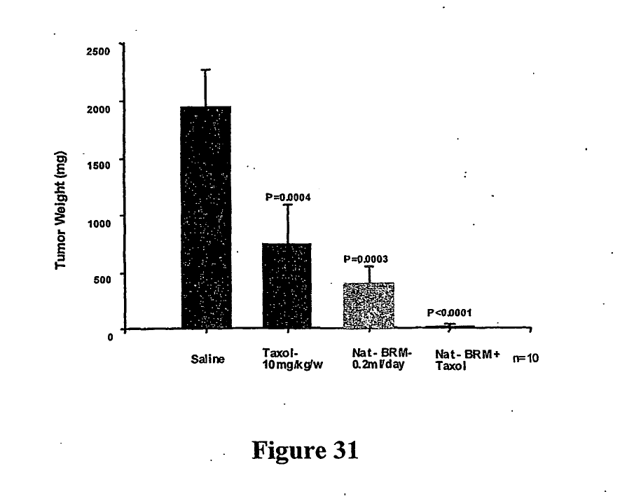

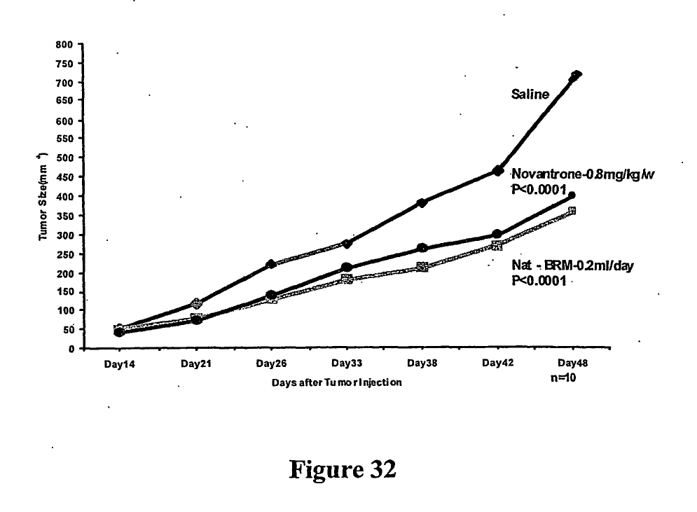

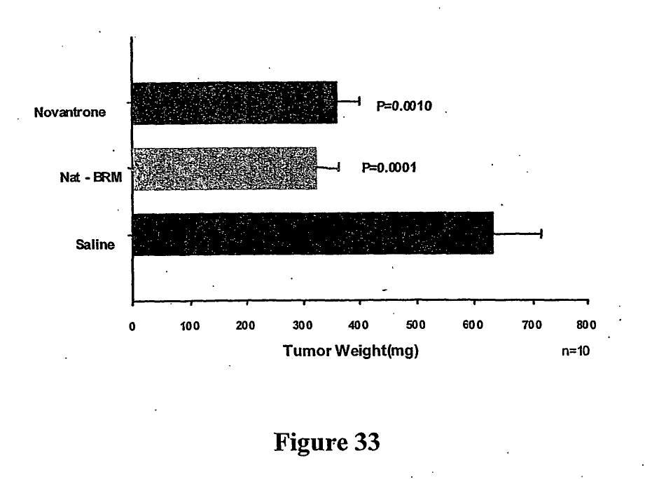

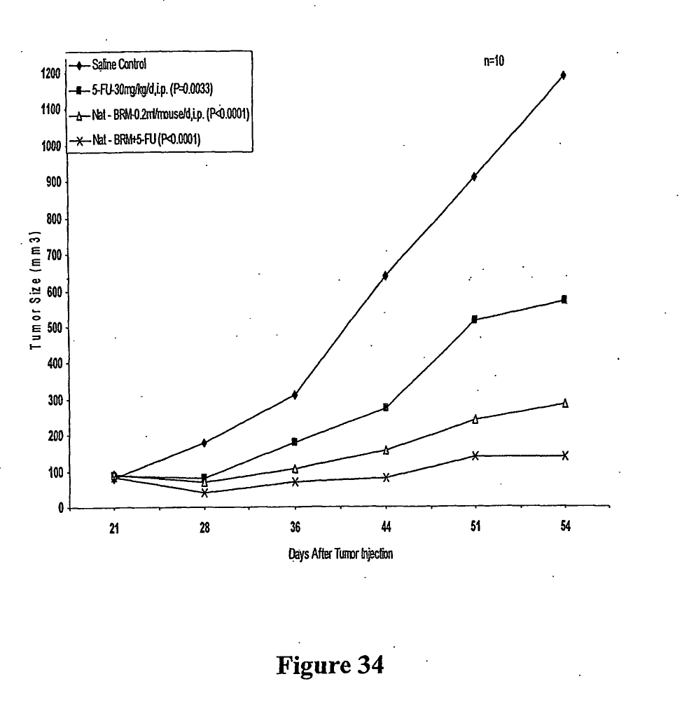

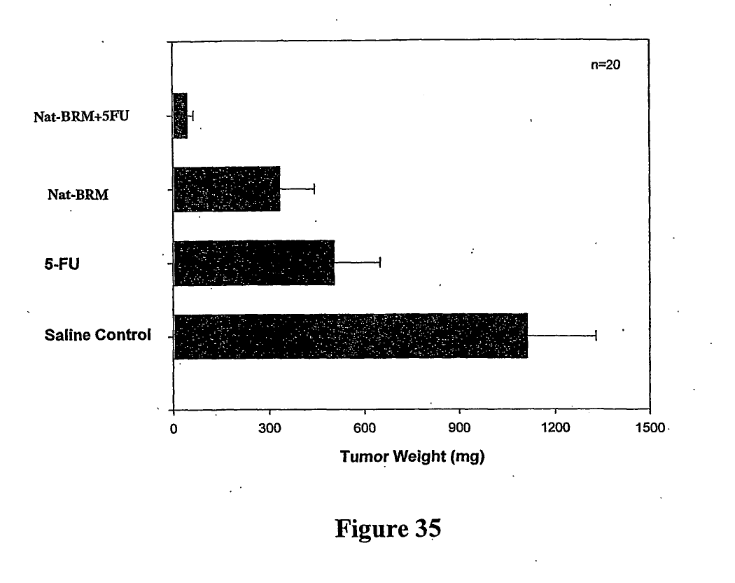

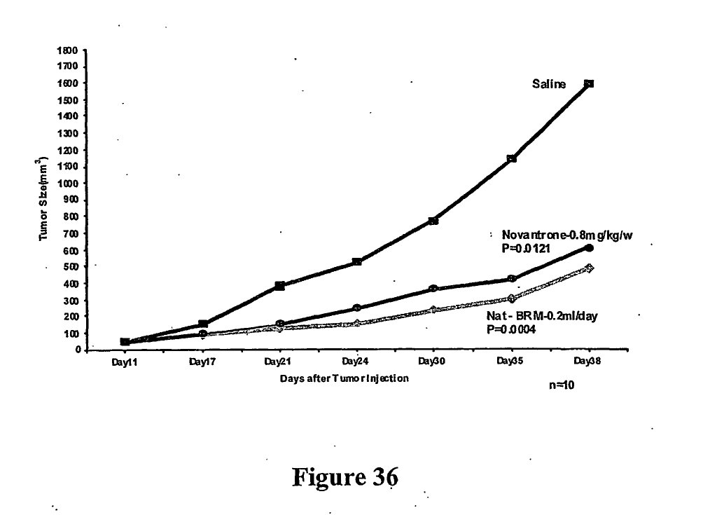

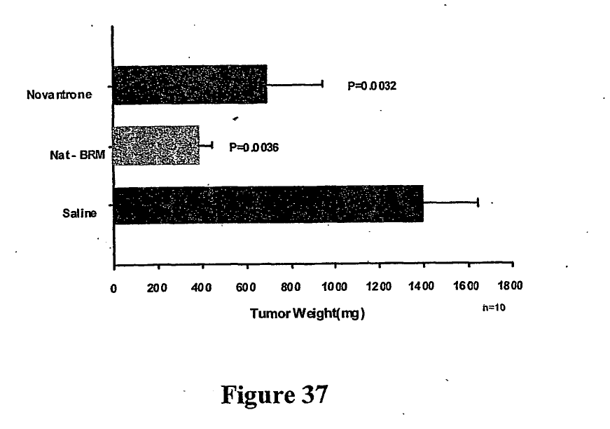

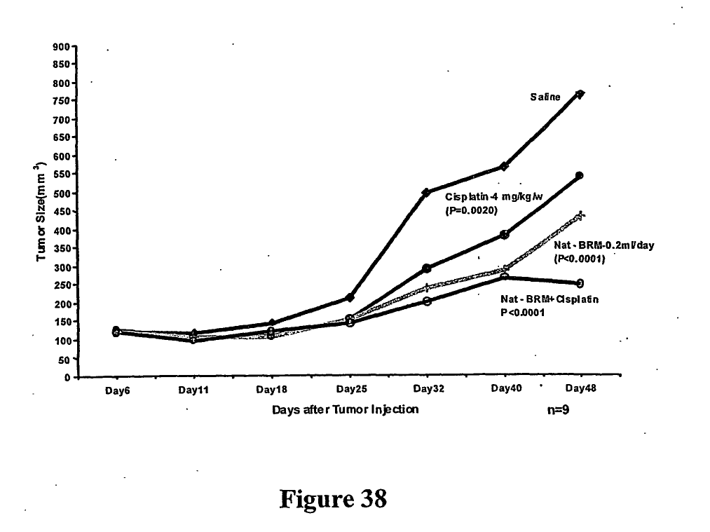

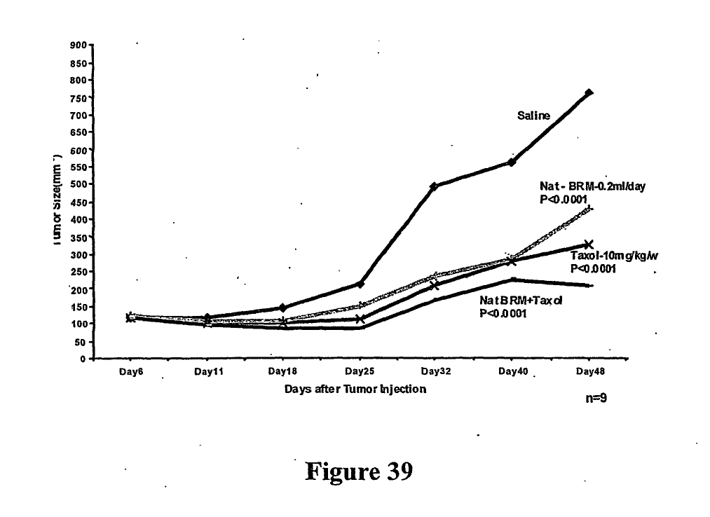

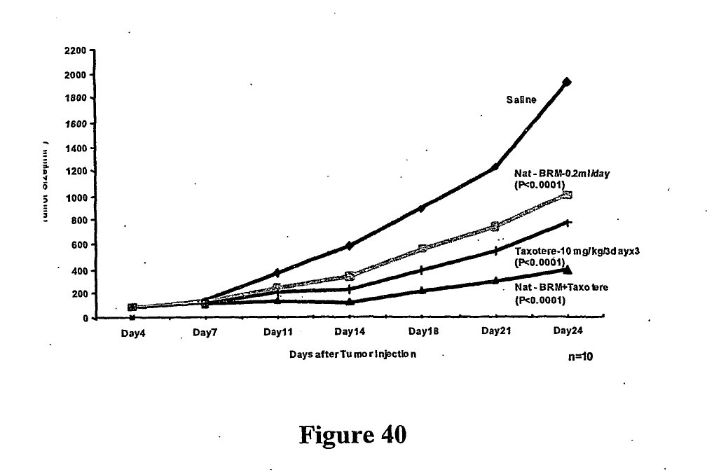

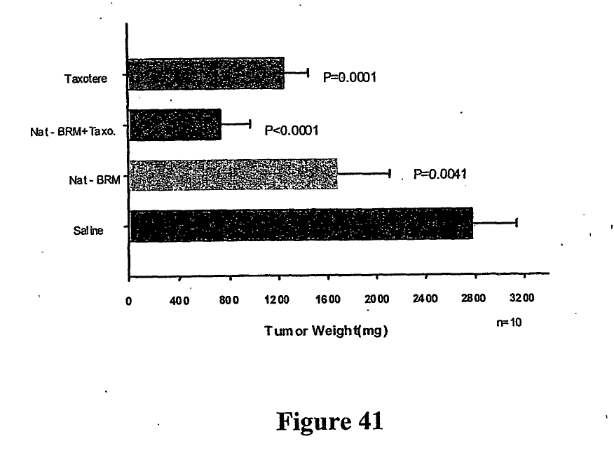

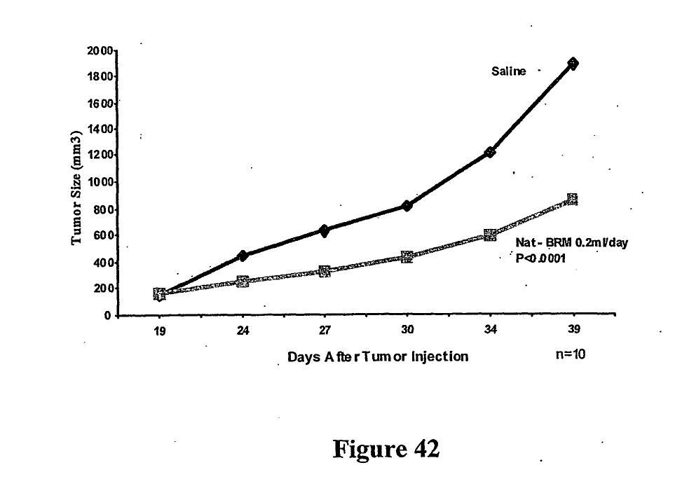

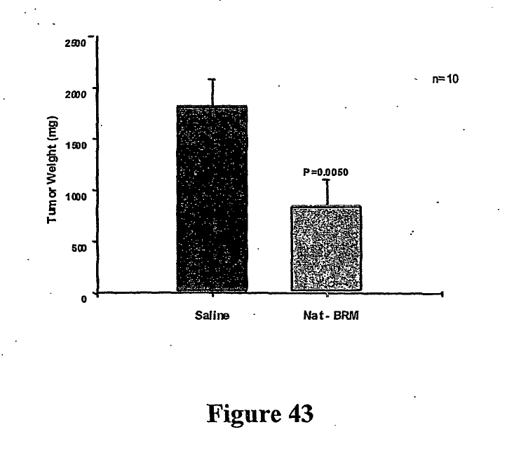

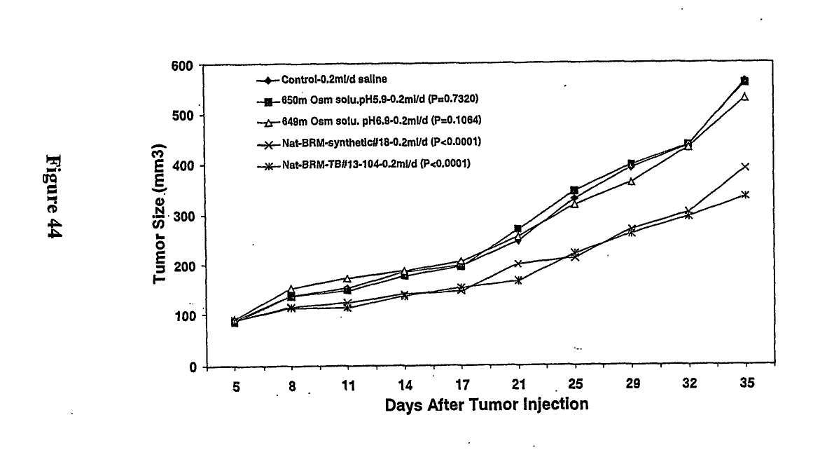

The results of the mouse xenograft experiments outlined in the table above are shown in FIGS. 20-43. Nat-BRM treatments always resulted in significant delay of tumor growth compared to saline control. Where a chemotherapeutic drug treatment group was included, the delay in tumor growth achieved with Nat-BRM was typically superior to the inhibitory effects observed with the chemotherapeutic drug. As indicated in the above table, total regression of the tumor was also observed in some of the animals, when the animals were treated with a BRM composition alone or with a combination of the Nat-BRM composition and a chemotherapeutic drug was used. In the remaining animals treated with a combination, significantly enhanced antitumor effects were observed.

The efficacy of the combinations of the invention can also be determined experimentally using other protocols to study animal models grafted with cancerous cells. The animals subjected to the experiment can be grafted with a tumor fragment, and the graft may be placed subcutaneously. In the case of the treatment of advanced tumors, tumors are allowed to develop to the desired size, animals having insufficiently developed tumors being eliminated. Animals not bearing tumors may also be subjected to the same treatments as the tumor-bearing animals in order to b able to dissociate the toxic effect from the specific effect on the tumor. Treatment generally begins 3 days to 4 weeks after grafting, depending on the type of tumor, and the animal are observed and animal weight change recorded, and the tumors measured regularly, for example daily, or 2 or 3 times per week until the tumor reaches a defined size (e.g. 2 g in a mouse), or until the animal dies if this occurs before hte tumor reaches 2 g. The animals are autopsied when sacrificed. To study leukemia, cancerous cells can be injected intravenously. Antitumor activity is determined by the increase in the survival time of the treated animals relative to the controls. The efficacy of the treatment with the combination of the invention is assessed in terms of changes in the mean survival time of the animal. Alternative methods of assessing efficacy, and therapeutic synergy, can also be used.

These animal models are recognized in the art to be predictive tests for anticancer effects in humans.

### Example 21

### In Vivo Evaluation of Efficacy of Syn-BRM

In addition to the in vivo xenograft models described in Examples 2-7 and Example 18, other animals models of disease can be used assess the effect of Syn-BRM. For example tumour growth may be assayed in mice carrying human solid tumour isografts introduced by fat pad injection. Other animal models of cancer include an experimental model of lymphoma and leukemia in mice (survival assay) which may be applied to, for example, Burkitts lymphoma (Non-Hodgkin's) (raji) or murine erythroleukemia (CB7 Friend retrovirus-induced), and also an experimental model of lung metastasis in mice as applied to human melanoma (C8161) or murine fibrosarcoma (R3).

Any of these or other in vitro or in vivo models may also be used to assess the effect of treatment with Syn-BRM in combination with various anticancer agents, such as chemotherapeutic drugs, radiation, a gene therapy and an antisense oligonucleotide.

### Example 22

### Evaluation of Syn-BRM Activities

A worker skilled in the art can produce Syn-BRM compositions, and assay Syn-BRM compositions for activities such as in vitro and/or in vivo monocyte and/or macrophage stimulation, modulation of tumor necrosis factor production and/or releases content of IL-1□, IL-1□, TNF, IL-6, IL-8, IL4, GM-CSF or IFN-gamma and endotoxin and cytotoxicity to human peripheral blood mononuclear cells, using the methods described in International Patent Application Serial No. PCT/CA94/00494, published Feb. 16, 1995 as WO 95/07089.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

TABLE 1
Biological Response Modifier Composition (Syn-BRM #1)

| | Concentration |
|---|---|
| Compound | (mg/L) |
| 3-Hydroxybutyric Acid | 560 |
| Acetic Acid | 480 |
| Lactic Acid | 105 |
| Urea | 270 |
| Creatinine | 1.5 |
| Creatine | 1 |
| Choline | 1 |
| Phosphoryl Choline | 1 |
| Taurine | 1 |
| Carnitine | 1 |
| Glycerol | 1 |
| Methyl Hydantoin | 1 |

Optionally containing:

| | |
|---|---|
| Phosphate | 15386 mg/L |
| Sodium | 9765 mg/L |
| Chloride | 9137 mg/L |

TABLE 2
Biological Response Modifier Composition (Syn-BRM #2)

| | Concentration |
|---|---|
| Compound | (mg/L) |
| 3-Hydroxybutyric Acid | 560 |
| Acetic Acid | 480 |
| Lactic Acid | 105 |
| Urea | 270 |
| Creatinine | 1.5 |
| Creatine | 1 |
| Choline | 1 |
| Phosphoryl Choline | 1 |
| Taurine | 1 |
| Carnitine | 1 |
| Glycerol | 1 |
| Methyl Hydantoin | 1 |

TABLE 3a
Biological Response Modifier Composition (Syn-BRM #3)

| Compound | mg/L |
|---|---|
| Urea | 302-930 |
| Acetic acid | 140-260 |
| Lactic acid | 33-260 |
| 3-hydroxybutyric acid | 43-80 |
| Choline | 17-29 |
| Creatinine | 13-23 |
| Taurine | 2.6-12 |
| Carnitine | 1.5-3 |
| Creatine | 1.1-1.6 |

TABLE 3b
Biological Response Modifier Composition (Syn-BRM #4)

| Compound | mg/L |
|---|---|
| NaCl | 14900 |
| Na₂HPO4 | 1390 |
| NaH₂PO4 | 780 |
| Urea | 815 |
| Acetic acid | 155 |
| Lactic acid | 125 |
| 3-hydroxybutyric acid | 57 |
| Formic acid | 40 |
| Creatinine | 22 |
| Choline | 20 |
| Taurine | 6.8 |
| Carnitine | 2.5 |
| Creatine | 1.4 |
| pH | Adjusted to ≅7.0 |
| Osmolarity | Adjusted to ≅650 mOsm |

TABLE 4
Proton Chemical Shift Assignments

| | Chemical | |
|---|---|---|
| Compounds | shifts (ppm) | Proton groups |
| 3- | 1.2 | CH₃ |
| hydroxybutyric | 2.31, 2.41 b | CH₂—CO |
| acid | 4.14 b | CH—O |
| lactic acid | 1.33 | CH₃ |
| | 4.12 b | CH—O |
| acetic acid | 1.93 | CH₃ |
| Creatine | 3.04 | CH₃—N |
| | 3.94 | CH₂—N |
| Creatinine | 3.05 | CH₃—N |
| | 4.07 | CH₂—N |
| Carnitine | 2.43 b | CH₂—N |
| | 3.23 | CH₃—N |
| | n | CH₂—CO; CH—O |
| Taurine | 3.28 | CH₂—N |
| | 3.44 b | CH₂—S |
| Choline | 3.22 | CH₃—N |
| | 3.54 b | CH₂—N |
| | 4.10 b | CH₂—O |
| Urea | 5.78 | NH₂—N |
| formic acid | 8.46 | CH—CO |
| Gly | 3.58 | αCH₂ |
| Ala | 1.49 | βCH₃ |
| | n | αCH |
| Glu | 2.06 b | βCH₂ |
| | 2.33 b | γCH₂ |
| | n | αCH |
| Gln | 2.14 b | βCH₂ |
| | 2.45 b | γCH₂ |
| | n | αCH |
| taurocholic | 0.89, 0.93, | C18, C19, C20, C22, |
| acid | 1.55, 2.04 b | respectively |
| | 2.21 b, 3.09, | C23, C25, C26, |
| | 3.52 b | respectively |
| betaine | 3.27 | (CH₃)₃—N |
| | 3.91 | CH₂—CO |
| glycolate | 3.96 | CH₃ |
| succinate | 2.41 | CH₂ |

TABLE 6
Inorganic analysis

| | Dalton | | | |
|---|---|---|---|---|
| | (408) | (407) | Imutec | |
| Parameters | 04002 ppm | 03202 ppm | BC0317 ppm | BC0318 ppm |
| Sodium | 5,990 | 6,515 | 5,960 | 5,855 |
| Phosphorus | 297 | 292 | 216 | 236 |
| Potassium | 244 | 311 | 277 | 276 |
| Magnesium | 1.20 | 1.00 | 0.95 | 1.05 |
| Calcium | 4.65 | 2.50 | 3.65 | 1.15 |
| Tungsten | N.D. | N.D. | N.D. | N.D. |
| Barium | N.D. | N.D. | N.D. | N.D. |
| Zinc | 1.00 | 3.00 | 6.00 | 3.75 |
| Nickel | N.D. | N.D. | N.D. | N.D. |
| Chromium | 0.05 | 0.20 | 0.20 | 0.20 |
| Strontium | N.D. | N.D. | N.D. | N.D. |
| Copper | N.D. | N.D. | N.D. | N.D. |
| Manganese | N.D. | N.D. | N.D. | N.D. |
| Molybdenum | N.D. | N.D. | 4.35 | 1.95 |
| Chloride | 9,950 | 11,380 | 9,715 | 10,460 |
| Sulphate | 88.7 | 90.2 | 109 | 111 |
| Phosphate | 909 | 894 | 662 | 724 |
| Fluoride | 72.0 | 58.0 | 52.2 | 63.5 |
| Bromide | 9.50 | 10.9 | 11.1 | 11.8 |
| Nitrate | 1.31 | N.D. | 3.40 | 1.20 |

N.D. = Not Detected

TABLE 7
Nat-BRM anti-tumor activity: human cancer xenoplants in mice

| | | | |
|---|---|---|---|
| | | | Combination |
| | | Nat-BRM | Therapy |
| Cancer type | Mouse | Mono- | (clinical-use |
| tumor cell line | Strain | therapy | drug) |

Skin Cancer

| | | | |
|---|---|---|---|
| Melanoma A2058 | CD-1 Nude | + | + (DTIC) |
| Melanoma C8161 | CD-1 Nude | + | + (DTIC) |
| Pancreatic | | | |
| Adenocarcinoma BxPc-3 | CD-1 Nude | + | + (Gemcitabine) |
| | | | + (5-FU) |
| Carcinoma SU 86.86 | CD-1 Nude | + | + (Gemcitabine) |
| | | | + (5-FU) |
| MIA-PaCa-2 | CD-1 Nude | + | + (Gemcitabine) |
| Breast | | | |
| Adenocarcinoma MDA- | CD-1 Nude | + | + (Taxol) |
| MB-231 | | | − (Doxorubicin) |
| Adenocarcinoma MVB-9 | CD-1 Nude | + | N/A |
| Ovarian | | | |
| Adenocarcinoma SK-OV-3 | CD-1 Nude | + | + (Taxol) |
| | | | * (Cisplatin) |
| Prostatic | | | |
| Carcinoma DU145 | SCID | + | − (Novantrone) |
| Carcinoma PC-3 | SCID | + | − (Novantrone) |
| Lung Cancer | | | |
| Small cell lung | SCID | + | + (Carboplatin) |
| carcinoma NCI-H209 | | | |
| Large cell lung | CD-1 Nude | +/− | +/− (Taxotere) |
| carcinoma NCI-H460 | | | |
| Colon | | | |
| Adenocarcinoma HT29 | CD-1 Nude | − | + (5-FU) |
| Lymphoma | | | |
| Non-Hodgkin's | SCID | − | N/A |
| lymphoma Raji | | | |

1. A synthetic biological response modifier composition comprising 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea, wherein said composition:
(a) stimulates or activates monocytes and macrophages; and/or
(a) modulates tumor necrosis factor production and/or release.

2. The composition of claim 1, wherein the composition may additionally comprise 40±14 mg/L formic acid.

3. A synthetic biological response modifier composition comprising 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea, 40±14 mg/L formic acid,
wherein said composition:
(a) stimulates or activates monocytes and macrophages; and/or
(a) modulates tumor necrosis factor production and/or release.

4. A pharmaceutical composition comprising:
a synthetic biological response modifier composition comprising 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea, wherein said composition:
(a) stimulates or activates monocytes and macrophages; and/or
(b) modulates tumor necrosis factor production and/or release; and
a pharmaceutically acceptable carrier or diluent.

5. (Canceled)

6. The use of the composition of any one of claims 1 to 3 to prepare a medicament for use in the treatment of cancer.

7. A combination comprising:
a composition according to any one of claims 1 to 3, wherein said composition:
(a) stimulates or activates monocytes and/or macrophages and/or;
(b) modulates tumor necrosis factor production and/or release; and
one or more anticancer agent(s), wherein said combination has therapeutic synergy or improves the therapeutic index in the treatment of cancer over the composition or the anticancer agent(s) alone.

8. The combination according to claim 7, wherein said anticancer agent(s) is selected from the group consisting of a chemotherapeutic drug, radiation, a gene therapy and an antisense oligonucleotide.

9. The combination according to claim 8, wherein at least one of said one or more anticancer agent(s) is a chemotherapeutic drug.

10. The combination of claim 9, wherein the chemotherapeutic drug is gemcitabine, 5-fluorouracil, dacarbazine, taxol, taxotere, cisplatin or mitoxantrone.

11. (Canceled)

12. (Canceled)

13. A pharmaceutical kit comprising:
a dosage unit of a synthetic biological response modifier composition and a pharmaceutically acceptable carrier wherein, the composition comprises 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea, wherein said composition:
(a) stimulates or activates monocytes and macrophages; and/or
(b) modulates tumor necrosis factor production and/or release; and a dosage unit of one or more chemotherapeutic drug(s).

14. A method for treating cancer in a mammal, comprising the step of administering to a mammal an effective amount of a synthetic biological response modifier composition comprising 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea, wherein said composition:
(a) stimulates or activates monocytes and macrophages; and/or
(b) modulates tumor necrosis factor production and/or release.

15. The synthetic biological response modifier composition according to any one of claims 1 to 3, wherein the composition may additionally comprise 14900 mg/L NaCl, 780 mg/L NaH₂PO₄, and 1390 mg/L Na₂HPO₄.

16. The synthetic biological response modifier composition according to any one of claims 1 to 3, wherein the pH of the composition is approximately 7.

17. The synthetic-biological response modifier composition according to any one of claims 1 to 3, wherein the osmolarity of the composition is approximately 650 mOsm.

18. The method according to claim 14, wherein said composition additionally comprises 40±14 mg/L formic acid.

19. The method according to claim 14, wherein said cancer is selected from the group consisting of: melanoma, lymphoma, pancreatic cancer, breast cancer, ovarian cancer, prostate cancer, lung cancer and colon cancer.

20. The method according to claim 14, wherein said cancer is a drug resistant cancer.

21. A method of activating natural killer (NK) cells in a mammal, comprising the step of administering to a mammal an effective amount of a synthetic biological response modifier composition comprising 57±20 mg/L 3-hydroxybutyric acid, 125±44 mg/L lactic acid, 155±54 mg/L acetic acid, 1.4±0.5 mg/L creatine, 22±8 mg/L creatinine, 2.5±0.9 mg/L carnitine, 6.8±2.4 mg/L taurine, 20±7 mg/L choline, 815±285 mg/L urea, wherein said composition:
(a) stimulates or activates monocytes and macrophages; and/or
(b) modulates tumor necrosis factor production and/or release.

22. The method according to claim 21, wherein said composition additionally comprises 40±14 mg/L formic acid.

23. A method of treating cancer comprising the step of administering to a mammal an effective amount of a combination comprising:
(i) the composition according to claim 1; and
(ii) one or more anticancer agent(s),
wherein said combination has therapeutic synergy or improves the therapeutic index in the treatment of cancer over the composition or the anticancer agent(s) alone.
